(19) United States
(12) Patent Application Publication
(10) Pub. No.: US 2019/0339285 A1
(43) Pub. Date: Nov. 7, 2019

(54) ION BEAM SCANNING OF BIOLOGICAL SAMPLES

(71) Applicant: IONpath, Inc., Menlo Park, CA (US)

(72) Inventors: David Stumbo, Pleasanton, CA (US); Rachel Finck, Menlo Park, CA (US)

(21) Appl. No.: 16/227,232

(22) Filed: Dec. 20, 2018

Related U.S. Application Data
(60) Provisional application No. 62/608,564, filed on Dec. 20, 2017.

(51) Int. Cl. G01N 33/68; H01J 49/00; G01N 33/483

(52) U.S. Cl. CPC G01N 33/6848; G01N 33/58; G01N 33/4833; H01J 49/0004

(57) ABSTRACT

The disclosure features methods of generating ions from a sample that include: exposing multiple regions of a biological sample on a substrate in succession to an ion beam to generate charged particles from each region, where the biological sample is labeled with at least one mass tag; for each exposed region, analyzing the plurality of charged particles to identify a deviation from a reference distribution of charged particles; and for each exposed region for which a deviation is identified, adjusting at least one exposure parameter of the ion beam based on the analysis of the plurality of charged particles to modify exposure of the sample to the ion beam.

### CROSS-REFERENCE TO RELATED APPLICATIONS

This application claims priority to U.S. Provisional Application No. 62/608,564, filed on Dec. 20, 2017, the entire contents of which are incorporated herein by reference.

### TECHNICAL FIELD

This disclosure relates to scanning of biological samples using an ion beam, and to determining mass spectrometry information for the samples based on ion beam exposure.

### BACKGROUND

Immunohistochemistry methods have been used to visualize protein expression in biological samples such as tumor tissue biopsies. Such methods typically involve exposing a sample to antibodies coupled to fluorescent moieties or enzyme reporters that generate colored pigments. Analysis of spectral images of the tagged sample yields information that can be used to assess protein expression levels and co-expression events. A variety of samples can be analyzed using such methods, including formalin-fixed, paraffin-embedded tissue sections.

### SUMMARY

This disclosure features multiplexed ion beam imaging methods for analyzing protein expression and other biological events and structures in tissue samples. Samples are tagged with antibodies conjugated to mass tags such as lanthanide elements and then exposed to a beam of primary ions. The primary ions are incident on the sample and generate secondary ions based on the mass tags. Spatially- and mass-resolved analysis of the secondary ions from the sample can provide information about protein expression and other biological events at specific sample locations.

Because exposing a sample to the primary ion beam and collecting and analyzing secondary ions generated from the sample is a time-consuming process, improvements in scanning speed and data quality can be realized when diagnostic methods are used to ensure that only regions of the sample are exposed to the primary ion beam. This disclosure includes methods and systems that determine whether the primary ion beam is incident on the sample, and adjust the exposure parameters of the primary ion beam to maintain sample exposure and reduce or prevent exposure of other objects to the primary ion beam.

Ensuring that the primary ion beam exposes only portions of the sample, and reducing or avoiding exposure of other objects (such as a substrate supporting the sample) to the primary ion beam can provide a number of advantages. For example, by confining the primary ion beam to exposure of the sample, the overall scanning time can be reduced, as objects that would otherwise yield secondary ion signals that are unrelated to the sample are avoided. For high-resolution scanning over relatively large exposure patterns, the reduction in overall scanning time can be significant.

As another example, by confining the primary ion beam to exposure of the sample, ablation of materials that would otherwise generate spurious signals or cause distortions in signals of interest can be reduced or avoided. Some samples are mounted on a substrate to which a conductive coating is applied. Ablation of the conductive coating can lead to charging of the underlying substrate material, leading to disruption of the primary ion beam and, in some circumstances, perturbing the process of secondary ion generation from the sample. Spurious contributions to measured secondary ion signals arising from such events can be reduced or eliminated by confining the primary ion beam to exposing regions of the sample. Additionally, by reducing or avoiding ablation of the conductive coating, the coating used can be made thinner, thereby reducing material usage and costs associated with sample mounting and preparation.

As a further example, signals generated by secondary ions from objects other than the sample can, in some circumstances, be stronger than secondary ion signals from the sample, and can be detected within a range of mass-to-charge ratios that is of interest for the sample. As such, these spurious signals effectively reduce the dynamic range of the detection system for weaker signals of interest from the sample, as the total signal detection range for the detection system also covers the spurious signals. By confining the primary ion beam to exposure of the sample, spurious signal generation can be reduced or avoided, and a larger portion of the detection system's dynamic range can be assigned to detection of secondary ions from the sample.

In general, in a first aspect, this disclosure features methods of generating ions from a sample, the methods including: exposing multiple regions of a biological sample on a substrate in succession to an ion beam to generate charged particles from each region, where the biological sample is labeled with at least one mass tag; for each exposed region, analyzing the plurality of charged particles to identify a deviation from a reference distribution of charged particles; and for each exposed region for which a deviation is identified, adjusting at least one exposure parameter of the ion beam based on the analysis of the plurality of charged particles to modify exposure of the sample to the ion beam.

Embodiments of the methods can include any one or more of the following features.

The at least one mass tag can include at least one antibody-conjugated lanthanide element.

Analyzing the plurality of charged particles can include measuring signal peaks corresponding to the charged particles and determining information associated with at least some of the signal peaks. The information can correspond to quantitative information featuring at least one of a peak amplitude, a peak area, and a charged particle count associated with each of the at least some of the signal peaks. Alternatively, or in addition, the information can include at least one of a mass-to-charge ratio and a quantity related to a mass-to-charge ratio associated with each of the at least some of the signal peaks. Alternatively, or in addition, the information can include an identity of a charged particle associated with each of the at least some of the signal peaks.

The methods can include, for each exposed region, identifying that a deviation from the reference distribution exists if the quantitative information differs from the reference distribution. The methods can include, for each exposed region, identifying that a deviation from the reference distribution exists if an element of the quantitative information exceeds a threshold value of the reference distribution. The methods can include, for each exposed region, identifying that a deviation from the reference distribution exists if the charged particles comprise ions generated from the substrate. The ions generated from the substrate can include silicon ions.

The methods can include, for each exposed region, identifying that a deviation from the reference distribution exists if the charged particles comprise ions generated from a coating on the substrate. The ions generated from the coating can include at least one type of ions selected from the group consisting of gold ions, tantalum ions, titanium ions, chromium ions, tin ions, and indium ions.

The methods can include, for each exposed region, identifying that a deviation from the reference distribution exists if an element of the quantitative information is less than a threshold value of the reference distribution. The methods can include, for each exposed region, identifying that a deviation from the reference distribution exists if the charged particles comprise ions generated from the sample. The ions generated from the sample can include at least one type of lanthanide ion.

The methods can include, for each exposed region, identifying that a deviation from the reference distribution exists if the information differs from the reference distribution. The methods can include, for each exposed region, identifying that a deviation from the reference distribution exists if a peak associated with a type of charged particle generated from the region is not present in the reference distribution. The peak can be associated with ions generated from the substrate. The ions generated from the substrate can include silicon ions. The peak can be associated with ions generated from a coating on the substrate. The ions generated from the coating can include at least one of gold ions, tantalum ions, titanium ions, chromium ions, tin ions, and indium ions.

The methods can include, for each exposed region, identifying that a deviation from the reference distribution exists if a peak associated with a type of charged particle is present in the reference distribution, but not among the peaks measured for the region. The peak can be associated with ions generated from the sample. The ions generated from the sample can include at least one type of lanthanide ion.

Adjusting at least one exposure parameter of the ion beam can include terminating exposure of the region to the ion beam. Terminating exposure of the region to the ion beam can include directing the ion beam away from the region. Terminating exposure of the region to the ion beam can include directing the ion beam to be incident on a beam blocking element.

Adjusting at least one exposure parameter of the ion beam can include reducing a dwell time of the ion beam on the region in a subsequent exposure of the region to the ion beam. The dwell time can be reduced by 90% or more (e.g., by 99.999% or more).

Adjusting at least one exposure parameter of the ion beam can include reducing an ion current of the ion beam during exposure of the region to the ion beam. The ion current can be reduced by 90% or more.

Adjusting at least one exposure parameter of the ion beam can include reducing an ion current of the ion beam during a subsequent exposure of the region to the ion beam. The ion current can be reduced by 90% or more (e.g., 99.999% or more).

The charged particles can include secondary electrons generated from one or more of the sample and the substrate, and analyzing the plurality of charged particles can include measuring a signal peak associated with the secondary electrons and determining quantitative information about a secondary electron yield from the signal peak, and comparing the quantitative information to a threshold value for the secondary electron yield from the reference distribution to determine whether a deviation from the reference distribution exists.

The charged particles can include ions generated from the sample, and analyzing the plurality of charged particles can include measuring one or more signal peaks associated with the ions generated from the sample and determining quantitative information about a total ion yield from the sample from the one or more signal peaks, and comparing the quantitative information to a threshold value for the total ion yield from the reference distribution to determine whether a deviation from the reference distribution exists.

A set of spatial locations of the multiple regions and exposure parameters of the ion beam at each spatial location can define a first exposure sequence for the sample, and the methods can include adjusting the at least one exposure parameter of the ion beam for each exposed region for which a deviation is identified to generate a second exposure sequence for a subsequent exposure of the sample to the ion beam. The second exposure sequence can include fewer spatial locations on the sample than the first exposure sequence. All spatial locations of the second exposure sequence can be common to the first exposure sequence. The second exposure sequence can include, at one or more spatial locations common to the first exposure sequence, ion beam dwell times that are reduced relative to corresponding dwell times of the first exposure sequence. The second exposure sequence can include, at one or more spatial locations common to the first exposure sequence, ion beam currents that are reduced relative to corresponding ion beam currents of the first exposure sequence.

The method can include, for each exposed region for which a deviation is identified, determining information about a thickness of the sample in the region based on the deviation from the reference distribution, and generating the second exposure sequence based on the thickness information. The methods can include determining information about an additional ion beam exposure dose for the region that will lead to elimination of the sample from the region. The methods can include determining information about an additional number of exposures of the region to the ion beam that will lead to elimination of the sample from the region.

The at least one antibody-conjugated lanthanide element can include at least one element selected from the group consisting of lanthanum, neodymium, samarium, gadolinium, erbium, ytterbium, and dysprosium. The biological sample can be labeled with multiple, different mass tags, and each of the mass tags can include an antibody-conjugated lanthanide element, e.g., each of which can be selected from the group consisting of lanthanum, neodymium, samarium, gadolinium, erbium, ytterbium, and dysprosium.

The biological sample can include a tissue sample (e.g., tumor tissue, formalin-fixed, paraffin-embedded tissue). The biological sample can include an array of single cells.

Embodiments of the methods can also include any of the other features disclosed herein, including combinations of features disclosed in different embodiments, unless expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

In general, method steps described herein and in the claims can be performed in any order, except where expressly prohibited or logically inconsistent. It should be noted that describing steps in a particular order does not mean that such steps must be performed in the described order. Moreover, the labeling of steps with identifiers does not impose an order on the steps, or imply that the steps must be performed in a certain sequence. To the contrary, the steps disclosed herein can generally be performed in any order except where noted otherwise.

The details of one or more embodiments are set forth in the accompanying drawings and the description below. Other features and advantages will be apparent from the description, drawings, and claims.

### DESCRIPTION OF DRAWINGS

FIG. 1 is a schematic diagram of an example of a multiplexed ion beam imaging system.

FIG. 2 is a schematic cross-sectional diagram of a sample on a substrate.

FIG. 3 is a schematic cross-sectional diagram of a sample with one or more conformal coating layers.

FIG. 4A is a schematic diagram showing an example of an ion beam exposure pattern on a sample.

FIG. 4B is a schematic diagram showing another example of an ion beam exposure pattern on a sample in which rows of the exposure pattern are offset.

FIG. 4C is a schematic diagram showing an example of a radial ion beam exposure pattern.

FIG. 5 is a schematic diagram showing an example of a portion of ion beam optics for a multiplexed ion beam imaging system.

FIG. 6 is a flow chart showing a series of example steps for adjusting ion beam exposure parameters based on measured secondary ion signals.

FIG. 7 is a schematic diagram showing representative ion counts/currents measured as a function of mass-to-charge ratio (m/z).

FIG. 8A is a schematic diagram showing measured secondary ion signals for a sample 150 as a function of mass-to-charge ratio (m/z).

FIG. 8B is a schematic diagram showing a reference distribution of charged particle peaks for a sample.

FIG. 9A is a schematic diagram of a reference distribution of charged particle peaks for another sample.

FIG. 9B is a schematic diagram showing measured secondary ion signals for the sample of FIG. 9A.

FIG. 10A is a schematic diagram showing a reference distribution of charged particle peaks for a further sample.

FIG. 10B is a schematic diagram showing measured secondary ion signals for the sample of FIG. 10A.

FIG. 11 is a schematic diagram showing an example of a modified ion beam exposure pattern on a sample.

FIGS. 12A-12C are plots showing time-dependent measurements of secondary ion signals corresponding to several different types of secondary ions.

FIG. 13A shows a set of plots of instantaneous measured ion counts and cumulative measured ion counts for 8 different spatial locations within a sample exposed to a primary ion beam, with a dwell time at each location of 0.25 ms.

FIG. 13B shows a set of plots of instantaneous measured ion counts and cumulative measured ion counts for 8 different spatial locations within a sample exposed to a primary ion beam, with a dwell time at each location of 1 ms.

FIG. 13C shows a set of plots of instantaneous measured ion counts and cumulative measured ion counts for 8 different spatial locations within a sample exposed to a primary ion beam, with a dwell time at each location of 4 ms.

FIG. 14 shows a set of plots of instantaneous measured ion counts and cumulative measured ion counts for 8 different spatial locations within a sample exposed to the primary ion beam, with changepoints for each of the instantaneous plots and cumulative plots shown as vertical lines.

FIGS. 15A-15C are histograms showing instantaneous Au ion counts at a first changepoint, cumulative Au ion counts at a first changepoint, and depth values at a first changepoint for measured ion signals from a calibration sample.

FIGS. 16A and 16B show images of two different samples, each constructed from measured secondary ion signals filtered according to a universal instantaneous threshold value.

FIGS. 16C and 16D show images of the same samples as in FIGS. 16A and 16B, respectively, each constructed from measured secondary ion signals filtered according to a universal cumulative threshold value.

Like reference symbols in the various drawings indicate like elements.

### DETAILED DESCRIPTION

### (i) Introduction

Multiplexed visualization of protein expression and other biochemical moieties and structures allows researchers to identify important correlations between biological functional events. Visualization of protein expression can be used to assess malignancies in excised tissue samples as part of a diagnostic work-up, and in particular, to provide important information about signaling pathways and correlated structural development in tumor tissue.

Conventional multiplexed immunohistochemical techniques for visualizing protein expression typically rely on optical detection of fluorescence emission from a sample that has been labeled with multiple antibody-conjugated fluorophores. The conjugated fluorophores bind specifically to corresponding antigens in the sample, and imaging of fluorescence emission from the sample is used to assess the spatial distribution of the fluorophores. For samples in which antigen concentrations are relatively low, signal amplification (e.g., using multivalent, enzyme-linked secondary antibodies) can be used to aid visualization. However, the use of signal amplification techniques can compromise quantitative information (e.g., antigen concentration information) that might otherwise be extracted from sample images.

In conventional multiplexed immunohistochemical visualization techniques, other constraints can also be encountered. Optical detection and separation of spectral signatures of multiple fluorophores is a complex problem, particularly where the fluorescence spectra of the fluorophores exhibit significant overlap. Without robust discrimination between spectral signatures of the fluorophores, important expression-related information is not uncovered. Further, such techniques often rely on primary antibodies generated in dissimilar host species. These factors can limit the utility of conventional multiplexed immunohistochemical visualization techniques for predictive biomarker development and clinical diagnostics.

This disclosure features methods for performing multiplexed visualization of antigens and other biochemical structures and moieties in biological samples using secondary ion mass spectrometry. Structure-specific antibodies are conjugated to specific mass tags, typically in the form of metallic elements (e.g., lanthanide elements). When a sample is exposed to the conjugated antibody-mass tag labels, the labels bind to corresponding antigens. Exposure of the labeled sample to a primary ion beam liberates secondary ions corresponding to the conjugated mass tags from the labeled sample. Performing spatially-resolved detection of the secondary ions that are generated from the sample allows direct visualization of the localization of specific antigens in the sample, and extraction of quantitative information (e.g., antigen concentration) as a function of spatial location. This information can be combined with other structural information (e.g., information about tumor margins, cell types/morphologies) to develop a detailed assessment of tumor viability and progression in the sample.

The methods disclosed herein, which are referred to as multiplexed ion beam imaging (MIBI) methods, can be used to resolve spatial distributions of relatively large numbers of mass tags applied to samples. For example, visual and quantitative assessment of up to 100 different mass tags in a single sample are possible. Depending upon the nature of the mass tags applied to the sample, sensitivities in the parts-per-billion range can be achieved with a dynamic range of approximately 10⁵. Imaging resolution is typically comparable to optical microscopy at high magnification.

The following sections of this disclosure describe examples of systems for multiplexed ion beam imaging, components of the systems, and methods for performing multiplexed ion beam imaging and sample preparation.

### (ii) Multiplexed Ion Beam Imaging Systems

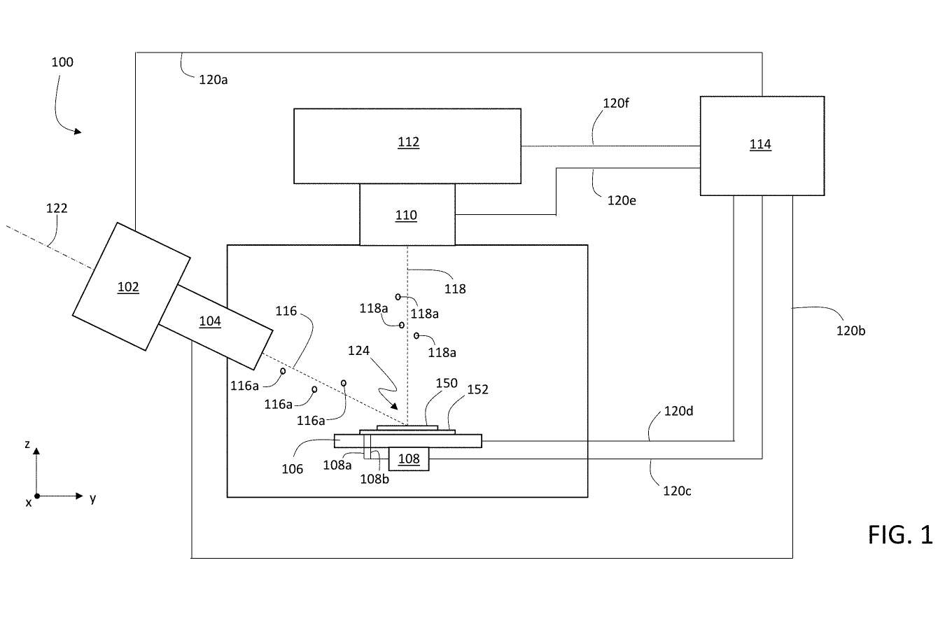

FIG. 1 is a schematic diagram showing an example system 100 for multiplexed ion beam imaging. System 100 includes an ion beam source 102, ion beam optics 104, a stage 106, a voltage source 108, ion collecting optics 110, and a detection apparatus 112. Each of these components is connected to a controller 114 via signal lines 120a-120f. During operation of system 100, controller 114 can adjust operating parameters of each of ion beam source 102, ion beam optics 104, stage 106, voltage source 108, ion collecting optics 110, and detection apparatus 112. Further controller 114 can exchange information with each of the foregoing components of system 100 via signal lines 120a-120f.

During operation, ion beam source 102 generates an ion beam 116 that includes a plurality of primary ions 116a. Ion beam 116 is incident on a sample 150 that is positioned on stage 106. Optionally, in certain embodiments, voltage source 108 applies an electrical potential to a substrate 152 that supports sample 150. Primary ions 116a in ion beam 116 interact with sample 150, generating secondary ions 118a as a secondary ion beam 118. Secondary ion beam 118 is collected by ion collecting optics 110 and directed into detection apparatus 112. Detection apparatus 112 measures one or more ion counts corresponding to secondary ions 118a in secondary ion beam 118 and generates electrical signals corresponding the measured ion counts. Controller 114 receives the measured electrical signals from detection apparatus 112 and analyzes the electrical signals to determine information about secondary ions 118a and sample 150.

Controller 114 can adjust a wide variety of different operating parameters of the various components of system 100, and can transmit information (e.g., control signals) and receive information (e.g., electrical signals corresponding to measurements and/or status information) from the components of system 100. For example, in some embodiments, controller 114 can activate ion beam source 102 and can adjust operating parameters of ion beam source 102, such as an ion current of ion beam 116, a beam waist of ion beam 116, and a propagation direction of ion beam 116 relative to central axis 122 of ion beam source 102. In general, controller 114 adjusts the operating parameters of ion beam source 102 by transmitting suitable control signals to ion beam source 102 via signal line 120a. In addition, controller 114 can receive information from ion beam source 102 (including information about the ion current of ion beam 116, the beam waist of ion beam 116, the propagation direction of ion beam 116, and various electrical potentials applied to the components of ion beam source 102) via signal line 120a.

Ion beam optics 104 generally include a variety of elements that use electric fields and/or magnetic fields to control attributes of ion beam 116. In some embodiments, for example, ion beam optics 104 include one or more beam focusing elements that adjust a spot size of ion beam 116 at a location of incidence 124 of ion beam 116 on sample 150. In certain embodiments, ion beam optics 104 include one or more beam deflecting elements that deflect ion beam 116 relative to axis 122, thereby adjusting the location of incidence 124 of ion beam 116 on sample. Ion beam optics 104 can also include a variety of other elements, including one or more apertures, extraction electrodes, beam blocking elements, and other elements that assist in directing ion beam 116 to be incident on sample 150.

Controller 114 can generally adjust the properties of any of the foregoing elements via suitable control signals transmitted via signal line 120b. For example, controller 114 can adjust the focusing properties of one or more beam focusing elements of ion beam optics 104 by adjusting electrical potentials applied to the beam focusing elements via signal line 120b. Similarly, controller 114 can adjust the propagation direction of ion beam 116 (and the location of incidence 124 of ion beam 116 on sample 150) by adjusting electrical potentials applied to the beam deflection elements via signal line 120b. Further, controller 114 can adjust positions of one or more apertures and/or beam blocking elements in ion beam optics 104, and adjust electrical potentials applied to extraction electrodes in ion beam optics 104, via suitable control signals transmitted on signal line 120b. In addition to adjusting properties of ion beam optics 104, controller 114 can receive information from various components of ion beam optics 104, including information about electrical potentials applied to the components of ion beam optics 104 and/or information about positions of the components of ion beam optics 104.

Stage 106 includes a surface for supporting sample 150 (and substrate 152). In general, stage 106 can be translated in each of the x-, y-, and z-coordinate directions. Controller 114 can translate stage 106 in an of the above directions by transmitting control signals on signal line 120d. To effect a translation of the location of incidence 124 of ion beam 116 on sample 150, controller 114 can adjust one or more electrical potentials applied to deflection elements of ion beam optics 104 (e.g., to deflect ion beam 116 relative to axis 122), adjust the position of stage 106 via control signals transmitted on signal line 120d, and/or adjust both deflection elements of ion beam optics 104 and the position of stage 106. In addition, controller 114 receives information about the position of stage 106 transmitted along signal line 120d.

In some embodiments, system 100 includes a voltage source 108 connected to substrate 152 via electrodes 108a and 108b. When activated by controller 114 (via suitable control signals transmitted on signal line 120c), voltage source 108 applies an electrical potential to substrate 152. The applied electrical potential assists in the capture of secondary ion beam 118 from sample 150, as the electrical potential repels secondary ions 118a, causing the secondary ions to leave sample 150 in the direction of ion collecting optics 110.

As shown in FIG. 1, sample 150 is typically a relatively planar sample that extends in the x- and/or y-coordinate directions and has a thickness measured in the z-coordinate direction. The support surface of stage 106 likewise extends in the x- and y-coordinate directions.

Secondary ion beam 118 consisting of a plurality of secondary ions 118a is captured by ion collecting optics 110. In general, ion collecting optics 110 can include a variety of electric and magnetic field-generating elements for deflecting and focusing secondary ion beam 118. In addition, ion collecting optics 100 can include one or more apertures, beam blocking elements, and electrodes. As discussed above in connection with ion beam optics 104, controller 114 can adjust electrical potentials applied to each of the components of ion collecting optics 110 via suitable control signals transmitted on signal line 120e. Controller 114 can also adjust the positions of apertures, beam blocking elements, and other movable components of ion collecting optics 110 by transmitting control signals on signal line 120e. In addition, controller 114 can receive information about operating parameters (e.g., voltages, positions) of various components of ion collecting optics 110 on signal line 120e.

Ion collecting optics 110 direct secondary ion beam 118 into detection apparatus 112. Detection apparatus 112 measures ion counts or currents corresponding to the various types of secondary ions 118a in secondary ion beam 118, and generates output signals that contain information about the measured ion counts or currents. Controller 114 can adjust various operating parameters of detection apparatus 112, including maximum and minimum ion count detection thresholds, signal integration times, the range of mass-to-charge (m/z) values over which ion counts are measured, the dynamic range over which ion counts are measured, and electrical potentials applied to various components of detection apparatus 112, by transmitting suitable control signals over signal line 120f.

Controller 114 receives the output signals from detection apparatus that include information about the measured ion counts or currents on signal line 120f. In addition, controller 114 also receives operating parameter information for the various components of detection apparatus 112 via signal line 120f, including values of the various operating parameters discussed above.

Detection apparatus 112 can include a variety of components for measuring ion counts/currents corresponding to secondary ion beam 118. In some embodiments, for example, detection apparatus 112 can correspond to a time-of-flight (TOF) detector. In certain embodiments, detection apparatus 112 can include one or more ion detectors such as Faraday cups, which generate electrical signals when ions are incident on their active surfaces. In some embodiments, detection apparatus 112 can be implemented as a multiplying detector, in which incident ions enter an electron multiplier where they generate a corresponding electron burst. The electron burst can be detected directly as an electrical signal, or can be incident on a converter that generates photons (i.e., an optical signal) in response to the incident electrons. The photons are detected with an optical detector which generates the output electrical signal.

As discussed above, controller 114 is capable of adjusting a wide variety of operating parameters of system 100, receiving and monitoring values of the operating parameters, and receiving electrical signals containing information about secondary ions 118a (and other species) generated from sample 150. Controller 114 analyzes the electrical signals to extract the information about secondary ions 118a and other species. Based on the extracted information, controller 114 can adjust operating parameters of system 100 to improve system performance (e.g., m/z resolution, detection sensitivity) and to improve the accuracy and reproducibility of data (e.g., ion counts) measured by system 100. Controller 114 can also execute display operations to provide system users with images of sample 150 that show distributions of various mass tags within sample 150, and storage operations to store information relating to the distributions in non-volatile storage media.

### (iii) Sample Preparation

In general, the methods and systems disclosed are compatible with a wide variety of biological samples. Typically, sample 150 is a tissue sample extracted from a human or animal patient. Sample 150 can correspond to a sample of tumor tissue excised during biopsy, or another type of tissue sample retrieved via another invasive surgical or non-invasive procedure.

In some embodiments, sample 150 corresponds to a formalin-fixed, paraffin-embedded tissue sample. Such samples are commonly prepared during histological workup of biopsied tissue from cancer tumors and other anatomical locations.

In certain embodiments, sample 150 corresponds to an array of single cells on a substrate. The array can be naturally occurring, and correspond to a regularly occurring, ordered arrangement of cells in a tissue sample. Alternatively, the array of cells can be a product of sample preparation. That is, the sample can be prepared by manual or automated placement of individual cells on substrate 152 (e.g., in a series of wells or depressions formed in substrate 152) to form the cell array.

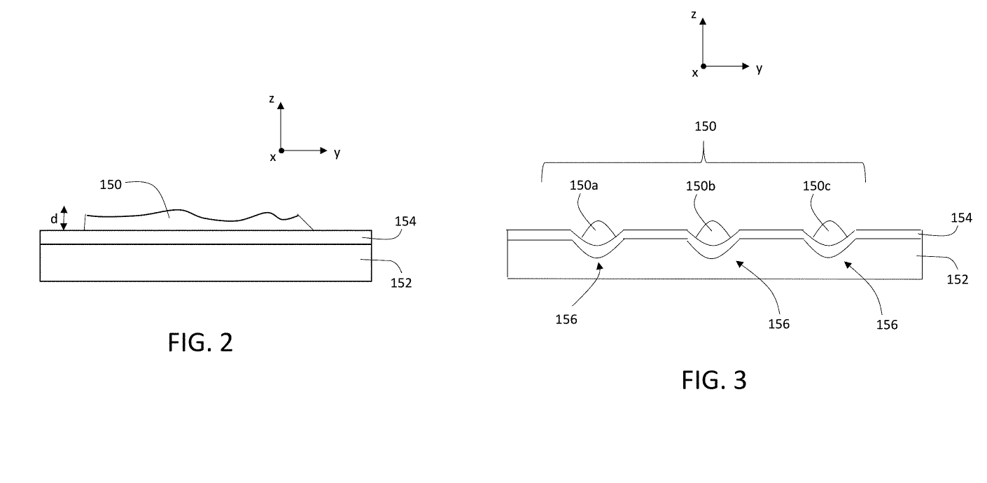

FIG. 2 is a schematic cross-sectional diagram of sample 150 on substrate 152. Positioned between substrate 152 and sample 150 in FIG. 2 is an optional coating 154. When present, coating 154 can be electrically connected to voltage source 108 via electrodes 108a and 108b, as shown in FIG. 1.

Substrate 152 is typically implemented as a microscope slide or another planar support structure, and can be formed from a variety of materials including various types of glass, plastics, silicon, and metals.

Coating 154, if present, is typically formed of one or more metallic elements, or one or more non-metallic compounds of relatively high conductivity. Examples of metallic elements used to form coating 154 include, but are not limited to, gold, tantalum, titanium, chromium, tin, and indium. In certain embodiments, coating 154 can be implemented as multiple distinct coating layers, each of which can be formed as a separate layer of a metallic element or a separate layer of a relatively high conductivity, non-metallic compound.

As shown in FIG. 2, sample 150 is approximately planar and extends in the x- and/or y-coordinate directions, and has a thickness d measured in the z-coordinate direction. Depending upon the method of preparation of sample 150, the sample can have an approximately constant thickness d across the planar extent of the sample parallel to the x-y coordinate plane. Alternatively, many real samples corresponding to excised tissue have non-constant thicknesses d across the planar extent of the sample parallel to the x-y coordinate plane. In FIG. 2, sample 150—which is shown in cross-section—has a non-constant thickness d measured in the z-coordinate direction.

In general, the thickness d of sample 150 depends upon the method by which sample 150 is obtained and processed prior to mounting on substrate 152. Certain samples, for example, are microtome-sliced from larger blocks of tissue, and can have relatively constant thicknesses. As another example, certain samples are obtained directly via excision, and can have variable thicknesses. The thickness d of sample 150 can be from 500 nm to 500 microns (e.g., from 1 micron to 300 microns, from 1 micron to 200 microns, from 1 micron to 100 microns, from 10 microns to 100 microns).

In some embodiments, substrate 152 can also include one or more additional coating materials to facilitate adhesion of sample 150 to substrate 152. Where no coating 154 is present, the one or more additional coating materials can be applied directly to substrate 150, such that the additional coating materials form a layer positioned between sample 150 and substrate 152. Where coating 154 is present, the one or more additional coating materials can be applied atop coating 154, for example, such that the additional coating materials form a layer positioned between coating 154 and sample 150. Suitable additional coating materials to facilitate adhesion of sample 150 include, but are not limited to, poly-1-lysine.

FIG. 3 shows a schematic cross-sectional diagram of another sample 150 positioned on a substrate 152. Substrate 152 optionally includes one or more conformal coating layers 154 as discussed above. In addition, substrate 152 includes an array of wells 156 corresponding to depressions formed in a surface of substrate 152. Each of the wells 156 contains a portion 150a-150c of sample 150. In general, while substrate 152 includes three wells 156 containing three separate portions 150a-150c of sample 150 in FIG. 3, more generally substrate 152 can include any number of wells 156, and sample 150 can be apportioned among any one or more of the wells 156.

Wells 156 (and the portions of sample 150 distributed among wells 156) can generally arranged in a variety of patterns in substrate 152. For example, wells 156 can form a linear (i.e., one dimensional) array in substrate 152. Alternatively, wells 156 can be distributed along one dimension in the plane of substrate 152, with irregular spacings between some or all of the wells.

As another example, wells 156 can form a two-dimensional array in substrate 152, with regular spacings between adjacent wells in directions parallel to both the x- and y-coordinate directions in the plane of substrate 152. Alternatively, in either or both of the directions parallel to the x- and y-coordinate directions in the plane of substrate 152, at least some of wells 156 can be spaced irregularly.

Where wells 156 form a two-dimensional array in substrate 152, the array can take a variety of forms. In some embodiments, the array of wells 156 can be a square or rectangular array. In certain embodiments, the array can be a hexagonal array, a polar array having radial symmetry, or another type of array having geometrical symmetry in plane of substrate 152.

As discussed above, each of the portions 150a-150c of sample 150 can include one or more cells. During sample preparation, each portion 150a-150c can be dispensed or positioned in a corresponding well 156 of substrate 152 to form sample 150. For example, each portion 150a-150c of sample 150 can be dispensed into a corresponding well 156 as a suspension of cells in a liquid medium, and the liquid medium subsequently removed (e.g., by washing or heating) to leave the cells in each well 156.

In general, to facilitate various biochemical structural analyses of sample 150 such as protein expression, sample 150 is labeled with multiple mass tags. When sample 150 is exposed to primary ion beam 116, the mass tags are ionized and liberated from sample 150. The ionized mass tags correspond to secondary ions 118a and form secondary ion beam 118 emerging from sample 150. Analysis of the secondary ions 118a present in secondary ion beam 118 as a function of the location of incidence 124 of ion beam 116 on sample 150 by controller 114 yields a wealth of information about the biochemical structure of sample 150 at each of the locations of incidence 124.

A variety of different mass tags can be used in the systems and methods disclosed herein. In some embodiments, the mass tags correspond to metallic elements, and more specifically, to lanthanide elements. Lanthanide elements suitable for use as mass tags include, for example, lanthanum, cerium, praseodymium, neodymium, promethium, samarium, europium, gadolinium, terbium, dysprosium, holmium, erbium, thulium, ytterbium, and lutetium.

To apply the mass tags to sample 150, each of the mass tags is conjugated to a specific antibody that selectively binds to an antigen receptor in sample 150. In practice, solutions of each of the antibody-conjugated mass tags are prepared, and then sample 150 is labeled by exposing sample 150 to each of the mass tag solutions. Sample 150 is typically exposed to multiple mass tag solutions sequentially and/or in parallel so that sample 150 can be labelled with multiple, distinct mass tags.

To prepare suitable mass tag labeling solutions, various methods can be used. In certain embodiments, for example, solutions are prepared by following a sequence of three steps: loading of a polymer linking moiety with a mass tagging element (e.g., a lanthanide metal element), reduction of an antibody, and conjugation of the metal-loaded polymer to the antibody. In the first step, for example, a metal-chelating polymer is loaded with a specific metal element. To perform this step, the polymer can be suspended in Buffer 1 (available from IONpath, Menlo Park, Calif.), and the metal element of interest added. The mixture can then be incubated for 45 minutes at 37° C., and subsequently transferred to a filter (e.g., a 3 kDa molecular-weight cut off (MWCO) Amicon® spin filter, available from MilliporeSigma, Burlington, Mass.) that retains the polymer and enables removal of unbound metal tags by washing the polymer twice with Buffer 1.

The second step, which can be performed in parallel with the first step, is to prepare the antibody to receive the polymer. The antibody can be transferred to a filter (e.g., a 50 kDa MWCO Amicon® spin filter) that retains the antibody, and then washed twice with Buffer 2 (available from IONpath, Menlo Park, Calif.). Next, the antibody can be incubated with a reducing agent, such as 4 mM tris(2-carboxyethyl)phosphine (TCEP) for 30 minutes at 37° C. to partially reduce the antibody and expose sulfhydryl residues for conjugation with the maleimide-containing polymer. Following the incubation, the antibody can be washed twice with Buffer 3 (available from IONpath, Menlo Park, Calif.) using the same 50 kDa MWCO filter to remove the TCEP and prevent further reduction to the antibody.

In the third step, the metal-tagged polymer and reduced antibody can be combined and incubated for 60-90 minutes at 37° C. to conjugate the polymer to the antibody. Following this incubation, the mixture can be washed three times with Buffer 4 (available from IONpath, Menlo Park, Calif.) using the same 50 kDa MWCO filter, which retains the conjugated antibody and allows removal of unbound polymer. The concentration of the metal-tagged antibody can be determined by measuring optical absorbance at 280 nm (e.g., using a NanoDrop spectrofluorometer, available from ThermoFisher Scientific, Waltham, Mass.) for a solution of the metal-tagged antibody in Buffer 5 (available from IONpath, Menlo Park, Calif.), diluted to a concentration of between 200 μg/mL and 500 μg/mL, and stored at a temperature of 4° C.

For preparation of samples consisting of arrays of cells, as shown in FIG. 3, cells in suspension can be augmented with surface marker antibodies and incubated at room temperature for approximately 30 minutes. Following incubation, cells can be washed twice with the mass tag labeling solutions to label the cells. Individual aliquots of the labeled cells, diluted in PBS to yield a desired concentration of cells per unit volume (e.g., approximately 10⁷ cells/mL), can then be placed in wells 156 and allowed to adhere for approximately 20 minutes. The adhered cells can then be gently rinsed with PBS, fixed for approximately 5 minutes in PBS with 2% glutaraldehyde, and rinsed twice with deionized water. Samples can then be dehydrated via a graded ethanol series, air dried at room temperature, and stored in a vacuum dessicator for at least 24 hours prior to analysis.

For preparation of intact tissue samples, such as samples obtained from biopsy, tissue samples can be mounted on substrate 152. Following mounting, the samples can be baked at approximately 65° C. for 15 minutes, deparaffinized in xylene (if obtained from FFPE tissue blocks), and rehydrated via a graded ethanol series. The samples are then immersed in epitope retrieval buffer (10 mM sodium citrate, pH 6) and placed in a pressure cooker (available from Electron Microscopy Sciences, Hatfield, Pa.) for approximately 30 minutes. Subsequently, the samples are rinsed twice with deionized water and once with wash buffer (TBS, 0.1% Tween, pH 7.2). Residual buffer solution can be removed by gently touch the samples with a lint free tissue. The samples are then incubated with blocking buffer for approximately 30 minutes (TBS, 0.1% Tween, 3% BSA, 10% donkey serum, pH 7.2).

The blocking buffer is then removed and the samples are labeled overnight with the mass tag labeling solutions at 4° C. in a humidified chamber. Following labeling, the samples are rinsed twice in wash buffer, postfixed for approximately 5 minutes (PBS, 2% glutaraldehyde), rinsed in deionized water, and stained with Harris hematoxylin for 10 seconds. The samples are then dehydrated via graded ethanol series, air dried at room temperature, and stored in a vacuum dessicator for at least 24 hours prior to analysis.

For example, a breast tumor tissue section can be prepared for MIBI as follows. Tissue sections (e.g., of 4 μm thickness) can be cut from formalin-fixed, paraffin-embedded (“FFPE”) tissue blocks of human breast tumor using a microtome, mounted on poly-1-lysine-coated silicon substrate for MIBI analysis. In some embodiments, silicon-mounted sections can subsequently be baked at 65° C. for 15 min, deparaffinized in xylene, and rehydrated via a graded ethanol series. The sections can then be immersed in epitope retrieval buffer (10 mM sodium citrate, pH 6) and placed in a pressure cooker for 30 min (e.g., from Electron Microscopy Sciences, Hatfield, Pa.). In some embodiments, after the pressure cooker, the sections can be rinsed twice with dH₂O and once with wash buffer (e.g., buffer containing Tris-buffered saline (“TBS”), 0.1% Tween, pH 7.2). Residual buffer can be removed, for example, by gently touching the surface with a lint-free tissue. The sections can then be incubated with blocking buffer for 30 min (TBS, 0.1% Tween, 3% BSA, 10% donkey serum, pH 7.2). The sections can be rinsed twice in wash buffer, postfixed for 5 min (PBS, 2% glutaraldehyde), rinsed in dH₂O. Finally, the sections can be dehydrated via graded ethanol series, air dried at room temperature, and then stored in a vacuum desiccator for at least 24 hrs prior to imaging.

Antigen retrieval can be performed using a decloaking chamber (e.g., from Biocare Medical, Concord, Calif.) with citrate buffer at pH 6.0, 125° C. and pressure to 15 psi. Sections can be in the chamber for a total time of 45 min. Incubations with primary antibodies can be performed at room temperature overnight in a humidified chamber. Normal goat serum can be used for blocking. Biotinylated goat anti-rabbit (1:1000) can be the secondary antibody used with a Vectastain ABC Kit Elite. A Peroxidase Substrate Kit DAB (e.g., from Vector Labs, Burlingame, Calif.) can be used for amplification and visualization of signal, respectively. Tissues known to contain each assessed antigen can be used as positive controls.

It should be understood that the above preparative steps are merely provided as examples of methods for sample preparation, and that modifications to the above sequences of steps also yield samples that are suitably labeled with mass tags and prepared for MIBI analysis. In particular, modifications to be above sequences of preparative steps can be undertaken based on the nature of the samples (e.g., the type of tissue to which the samples correspond).

### (iv) Sample Scanning

To perform multiplexed ion beam imaging following suitable labeling of sample 150 with multiple mass tags, primary ion beam 116 is directed to multiple different locations of incidence 124 on sample 150. At each location 124, primary ion beam 116 generates secondary ions 118a that correspond to the antibody-conjugated mass tags bound to sample 150 at that location. The secondary ions 118a—which form secondary ion beam 118—are measured and analyzed to determine spatially resolved information about the biochemical structure of sample 150.

A variety of different primary ion beams 116 generated by ion source 102 can be used to expose sample 150. In some embodiments, for example, primary ion beam 116 consists of a plurality of oxygen ions (O⁻). For example, ion source 102 can be implemented as an oxygen duoplasmatron source, which generates primary ion beam 116.

To obtain spatially resolved information from sample 150, primary ion beam 116 is translated across sample 150 to multiple different locations of incidence 124. The multiple different locations of incidence form a two-dimensional exposure pattern of primary ion beam 116 in the plane of sample 150 (i.e., in a plane parallel to the x-y plane). In general, a wide variety of different exposure patterns can be used.

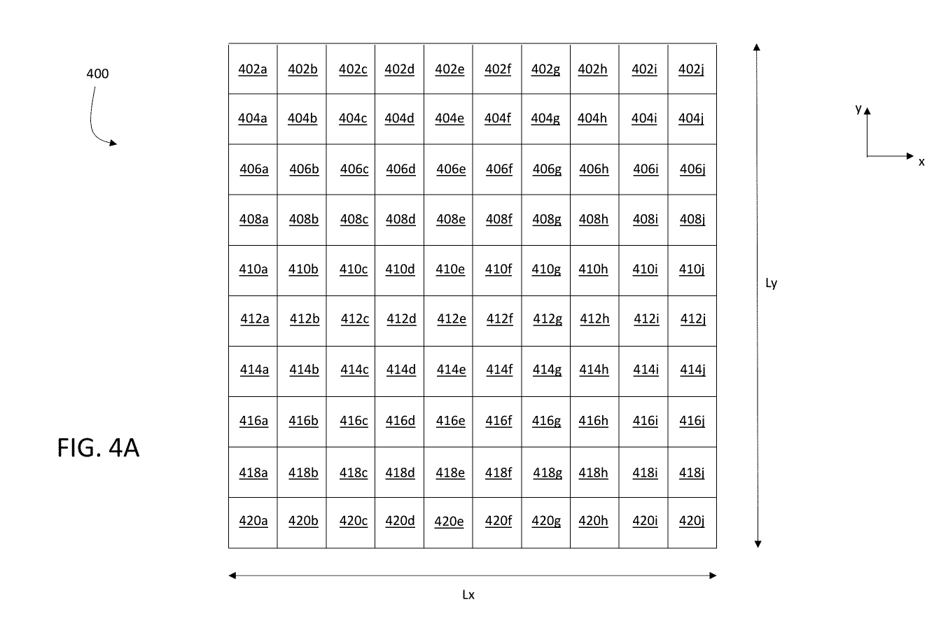

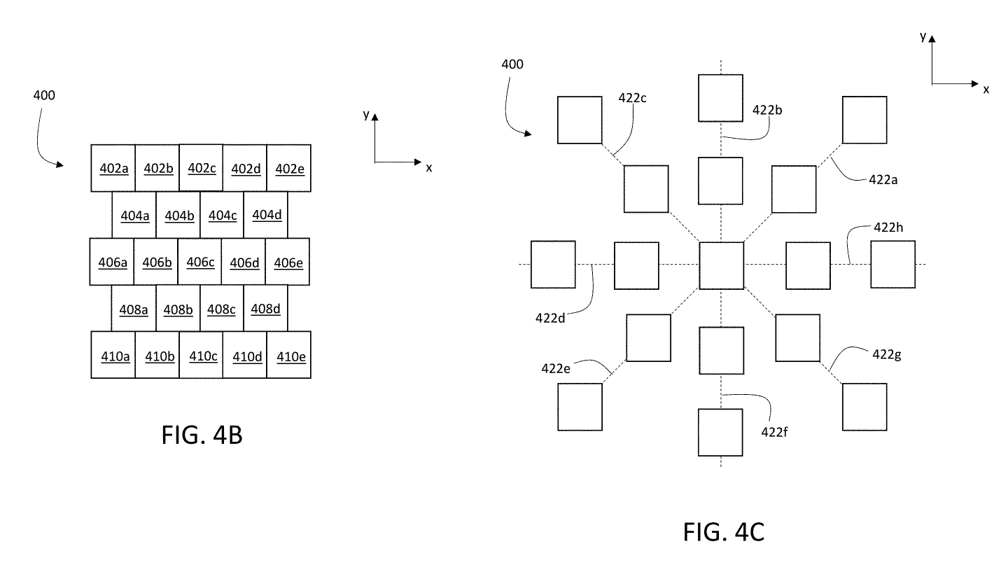

In some embodiments, for example, the exposure pattern corresponds to a square or rectangular array of locations of incidence 124 of primary ion beam 116 on sample 150. FIG. 4A is a schematic diagram showing a square array of locations of incidence 124 of primary ion beam 116 on sample 150, forming a square exposure pattern 400 on sample 150. Each row of exposure pattern 400 includes 10 distinct locations of incidence 124 of primary ion beam 116 on sample 150, spaced along the x-coordinate direction. Each column of exposure pattern 400 includes 10 distinct locations of incidence 124 of primary ion beam 116 on sample 150, spaced along the y-coordinate direction. In total, exposure pattern 400 includes 100 distinct locations of incidence 124 of primary ion beam 116.

In general, each row and column of exposure pattern 400 can include any number of distinct locations of incidence 124 of primary ion beam 116 on sample 150. For example, in some embodiments, each row and/or column of exposure pattern 400 includes 10 or more (e.g., 20 or more, 30 or more, 50 or more, 100 or more, 200 or more, 300 or more, 500 or more, 1000 or more) distinct locations of incidence 124 of primary ion beam 116.

To expose sample 150 to primary ion beam 116 according to exposure pattern, the different locations of incidence 124 constituting exposure pattern 400 can generally be visited in any order by primary ion beam 116. In some embodiments, however, the different locations of incidence 124 are visited in certain sequences. For example, the square exposure pattern 400 in FIG. 4A can be implemented such that primary ion beam 116 is scanned along each row of the exposure pattern in sequence. After visiting each location of incidence 124 in a single row in sequence (e.g., by translating primary ion beam 116 parallel to the x-coordinate direction), primary ion beam 116 is translated parallel to the y-coordinate direction to the next row in exposure pattern 400, and then visits each location of incidence 124 in the next row in sequence.

This example sequence of exposures corresponds to a pattern of raster-scanning of primary ion beam 116 on sample 150. As shown in FIG. 4A, locations 402a-402j are each visited in sequential order by primary ion beam 116, followed by locations 404a-404j in sequential order, and so on in sequence until the final row of locations 420a-420j is visited in sequential order.

Exposure pattern 400 includes a total of 100 distinct locations of incidence of primary ion beam 116 on sample 150. More generally, however, exposure pattern 400 can include any number of distinct locations of incidence of primary ion beam 116. In certain embodiments, for example, exposure pattern 400 includes 25 or more (e.g., 50 or more, 100 or more, 200 or more, 500 or more, 1000 or more, 5000 or more, 10000 or more, 20000 or more, 30000 or more, 50000 or more, 100000 or more, 200000 or more, 500000 or more) distinct locations of incidence of primary ion beam 116 on sample 150.

A maximum dimension of exposure pattern 400 measured in a direction parallel to the x-coordinate direction is Lx, and a maximum dimension of exposure pattern 400 measured in a direction parallel to the y-coordinate direction is Ly. In general, Lx and Ly are selected as desired according to the spatial dimensions of the portion of sample 150 to be analyzed. For example, in some embodiments, Lx and Ly can each independently be 25 microns or more (e.g., 50 microns or more, 100 microns or more, 200 microns or more, 300 microns or more, 400 microns or more, 500 microns or more, 700 microns or more, 1.0 mm or more, 1.5 mm or more, 2.0 mm or more, 2.5 mm or more, 3.0 mm or more, 5.0 mm or more).

Exposure pattern 400 in FIG. 4A is a square pattern. More generally, however, the exposure pattern formed by the set of locations of incidence 124 of primary ion beam 116 on sample 150 need not be square or rectangular. Two-dimensional exposure patterns having a variety of different shapes and spacings between locations of incidence of primary ion beam 116 can be implemented. FIG. 4B is a schematic diagram showing an exposure pattern 400 in which rows of the exposure pattern are offset spatially in the x-direction, forming an offset array. FIG. 4C is a schematic diagram showing a radial exposure pattern 400 in which individual locations of incidence of primary ion beam 116 are exposed in sequence along radial lines 422a-422h.

Returning to FIG. 4A, when sample 150 is exposed to primary ion beam 116 according to exposure pattern 400, the exposure can be implemented based on a single execution of exposure pattern 400 or based on multiple executions of exposure pattern 400. In other words, in some embodiments, sample 150 is exposed to primary ion beam 116 by directing primary ion beam 116 to visit each location in exposure pattern 400 once. In certain embodiments, sample 150 is exposed to primary ion beam 116 by directing primary ion beam 116 to visit each location in exposure pattern 400 multiple times. Typically, for example, after primary ion beam 116 has visited each location in exposure pattern 400 once, primary ion beam 116 follows a second exposure sequence in which the beam visits the locations in exposure pattern 400 a second time. Subsequent exposure sequences can be implemented in which primary ion beam 116 repeats the sequence of exposures defined by exposure pattern 400 as many times as desired.

In general, the accuracy and reproducibility of the ion counts/currents measured by detection apparatus 112 depends on number of secondary ions 118a generated by the interaction between primary ion beam 116 and sample 150. The number of secondary ions generated at each location of incidence 124 of primary ion beam 116 is in turn a function of the total primary ion dose at each location. As the primary ion dose increases, all other factors being held constant, the number of secondary ions generated also increases. As discussed above, the total dose of primary ions at each location of incidence 124 can be delivered via a single exposure to primary ion beam 116 at each location, or via multiple exposures to primary ion beam 116 at each location (i.e., by repeating exposure pattern 400).

In summary, as used herein, the term “exposure pattern”—examples of which are represented schematically by exposure patterns 400 in FIGS. 4A-4C—refers to the set of spatial locations of incidence of primary ion beam 116 on sample 150, as well as the set of dwell times (also referred to as exposure times), ion doses, ion beam currents, and other exposure parameters associated with each of the spatial locations of incidence of primary ion beam 116 on sample 150. In some embodiments, controller 114 maintains information corresponding to the exposure pattern in a volatile and/or non-volatile memory unit. During operation of system 100, controller 114 can modify the exposure pattern—by modifying the set of locations of incidence of primary ion beam 116 associated with the exposure pattern, and/or by modifying any of the exposure parameters associated with the set of spatial locations—in response to ion counts/currents measured by detection apparatus 112, and/or to adjust performance-related metrics for system 100 such as signal resolution, signal-to-noise ratio, and data reproducibility and/or accuracy.

In some embodiments, to control the location of incidence 124 of primary ion beam 116 on sample 150, controller 114 translates stage 106 in the x- and y-coordinate directions via control signals transmitted on signal line 120d. With primary ion beam 116 directed to a static location, motion of stage 106 in the x- and y-coordinate directions effects translations of sample 150 in the x- and y-coordinate directions relative to the location of primary ion beam 116, thereby moving the location of incidence 124 of primary ion beam 116.

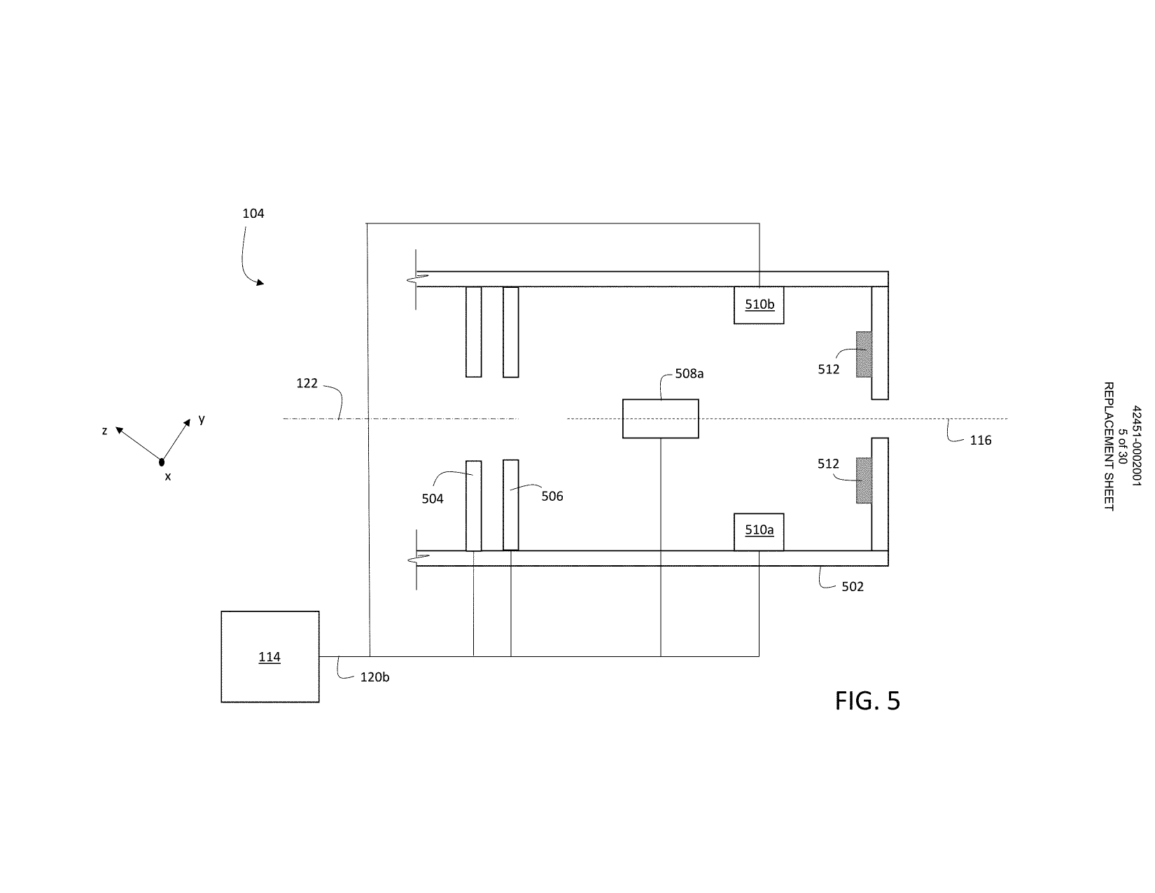

Alternatively, or in addition, in certain embodiments controller 114 adjusts one or more elements of ion beam optics 104 to translate the location of primary ion beam 116 on sample 150. FIG. 5 is a schematic diagram showing an example of a portion of ion beam optics 104. Ion beam optics 104 include a housing 502 that encloses a variety of components, including focusing elements 504 and 506 (implemented as annular electrostatic lenses), a first pair of deflection electrodes (only one of which, electrode 508a, is shown in FIG. 5 due to the perspective of the figure), and a second pair of deflection electrodes 510a and 510b. Ion beam optics 104 also include beam blocking elements 512.

Controller 114 is electrically connected to focusing elements 504 and 506, to the first pair of deflection electrodes (shown via a connection to electrode 508a in FIG. 5), and to the second pair of deflection electrodes 510a and 510b, via signal line 120b. Controller 114 adjusts electrical potentials applied to each of the elements to which it is connected by transmitting appropriate signals on signal line 120b.

During operation of system 100, primary ion beam 116 enters ion beam optics 104 through an aperture (not shown in FIG. 5) in housing 502, propagating nominally along central axis 122 of ion beam optics 104. By applying suitable electrical potentials to annular focusing elements 504 and 506, controller 114 adjusts the focal position of primary ion beam 116 along axis 122.

Controller 114 can adjust the location of incidence 124 of primary ion beam 116 on sample 150 by adjusting electrical potentials applied to the first and second pairs of deflection electrodes via control signals transmitted along signal line 120b. For example, by adjusting the electrical potentials applied to the first pair of deflection electrodes (electrode 508a and a cooperating second electrode not shown in FIG. 5), primary ion beam 116 is deflected in a direction parallel to the x-coordinate direction. Thus, to scan primary ion beam 116 in a direction parallel to the x-coordinate direction in an exposure pattern, controller 114 adjusts the electrical potentials applied to the first pair of deflection electrodes.

Similarly, by adjusting the electrical potentials applied to the second pair of deflection electrodes, 510a and 510b, a component of the resulting deflection of primary ion beam 116 is parallel to the y-coordinate direction. Accordingly, to scan primary ion beam 116 in a direction parallel to the y-coordinate direction in an exposure pattern, controller 114 adjusts the electrical potentials applied to the second pair of deflection electrodes.

To prevent primary ion beam 116 from being incident on sample 150, controller 114 can adjust the electrical potentials applied to either or both pairs of deflection electrodes to cause primary ion beam 116 to be intercepted by a beam blocking element. For example, by applying suitable electrical potentials to electrodes 510a and 510b, primary ion beam 116 can be deflected such that the beam is blocked by beam blocking elements 512 in ion beam optics 104. Beam blocking elements can also be positioned external to ion beam optics 104, and the electrical potentials applied to deflection electrodes adjusted to steer primary ion beam 116 to be incident on the external beam blocking elements.

### (v) Sample Monitoring and Exposure Adjustment

As discussed previously, samples obtained via excision and subsequently mounted on substrate 152 may have non-uniform thicknesses and may even contain holes and other significant irregularities. When primary ion beam 116 is scanned over such holes and irregularities, useful information about the sample is generally not obtained on account of the absence of mass tag-based secondary ions that are generated from these regions. Accordingly, measuring secondary ion signals from such regions for extended periods is generally wasteful, as it extends measurement times without providing useful information.

Furthermore, during exposure to primary ion beam 116, the exposed portions of sample 150 are ablated as secondary ions are generated from the mass tags labeling the sample. Thus, over time, even initially intact regions of a sample are degraded, after which time continued generation of secondary ions from such regions no longer provides useful information.

Scanning irregular and/or ablated regions of a sample with primary ion beam 116 can also have other undesirable effects. For example, when primary ion beam 116 is incident at a location 124 corresponding to a hole in sample 150 (either pre-existing or introduced via ablation of overlying tissue during prior exposure to primary ion beam 116), the primary ion beam 116 can ablate coating 154 on substrate 152. When coating 154 is partially ablated, the electrical potential applied to coating 154 by voltage source 108 can be less effective at directing secondary ions 118a away from sample 150 due to losses in conductivity within coating 154. Further, ablation of coating 154 exposes substrate 152 to primary ion beam 116. Typically, substrate 152 is formed from materials of relatively low electrical conductivity, such as glasses. When exposed to primary ion beam 116, such materials ionize and excess charge accumulates on the material surfaces, as the excess charge is not effectively conducted away. The accumulation of excess charges within substrate 152 can lead to scanning distortions when primary ion beam 116 is incident at other locations on sample 150; the accumulated charge can perturb the location of incidence 124 of primary ion beam 116, and the collection of secondary ions 118a by ion collecting optics 110 can also be impaired.

As another example, generation of secondary ions from coating 154 and/or substrate 152 can also reduce the portion of the dynamic signal detection range of detection apparatus 112 that is used for detection of secondary ions corresponding to mass tags generated from sample 150. In particular, secondary ion signals corresponding to coating 154 and/or substrate 152 can be relatively strong on account of the large volumes of material that form coating 154 and substrate 152. Accordingly, to detect such secondary ion signals without saturation, the dynamic signal detection range of detection apparatus 112 spans a relatively large range of signal intensities. Because secondary ion signals corresponding to mass tags that label a sample are typically significantly weaker, only a comparatively small fraction of the dynamic signal detection range is used to measure mass tag signals, resulting in reduced accuracy and signal intensity resolution.

To reduce or eliminate problems such as those described above arising from directing primary ion beam 116 to be incident at locations 124 where no sample is present, methods and systems that modify exposure parameters based on secondary ion signals (and, in some embodiments, other measured signals) are discussed herein. By analyzing the secondary ion signals, information about whether primary ion beam 116 is incident on a region corresponding to intact sample 150 or on a region that does not correspond to sample 150 is determined. One or more exposure parameters associated with the exposure of sample 150 to primary ion beam 116 can then be modified, particularly when it is determined that the location of incidence 124 of primary ion beam 116 does not correspond to an intact portion of sample 150.

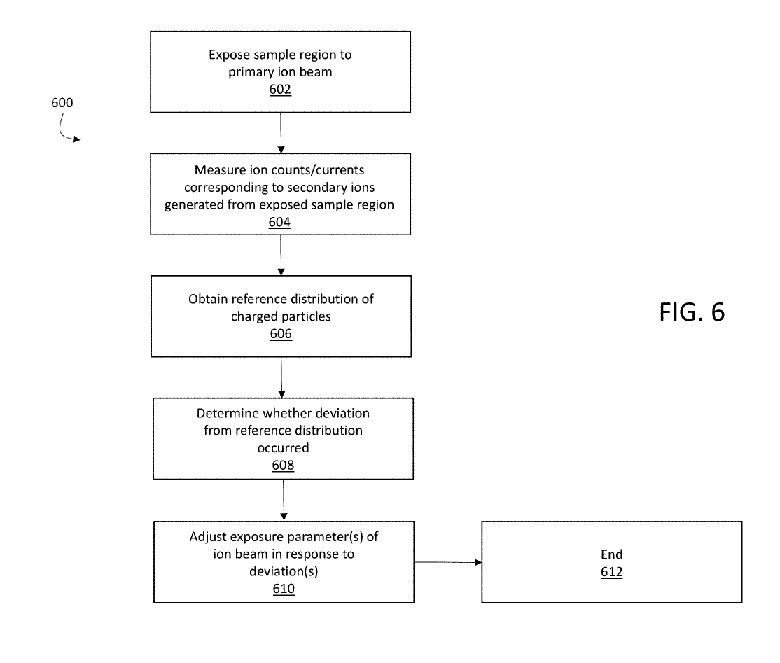

FIG. 6 is a flow chart 600 showing a series of example steps for adjusting ion beam exposure parameters based on measured secondary ion signals. In a first step 602, controller 114 activates ion source 102 to generate primary ion beam 116, and directs primary ion beam 116 using ion beam optics 104 to be incident on a spatial region that presumptively corresponds to a portion of sample 150. As discussed previously, when primary ions 116a of primary ion beam 116 are incident on a portion of sample 150, they interact with sample 150 to generate a plurality of secondary ions from sample 150.

Where sample 150 includes different types of antibody-conjugated mass tags bound to complementary antigens in the sample, at least some of the secondary ions 118a generated correspond to the individual mass tags. When the mass tags are implemented as lanthanide elements, at least some of the secondary ions 118a are lanthanide ions. Examples of such lanthanide ions include lanthanum ions, neodymium ions, samarium ions, gadolinium ions, erbium ions, ytterbium ions, and dysprosium ions.

Secondary ions 118a can also include other types of ions. In particular, when primary ion beam 116 is incident not on a portion of sample 150, but instead on a portion of coating 154, secondary ions 118a can include ions of the coating material. Secondary ions 118a corresponding to coating 154 can include, for example, gold ions, tantalum ions, titanium ions, chromium ions, tin ions, and indium ions.

Where primary ion beam 116 is not incident on a portion of sample 150 but is instead incident on a portion of substrate 152, secondary ions 118a can includes of the substrate material. An example of such secondary ions 118a generated from substrate 152 is silicon ions.

In the next step 604, ion counts/currents corresponding to the different types of secondary ions 118a generated in response to exposure by primary ion beam 116 are measured by detection apparatus 112. In general, the ion counts/currents are measured as a function of m/z. Information corresponding to the measured ion counts/currents is transmitted by detection apparatus 112 to controller 114, which analyzes the measured information. In some embodiments, for example, controller 114 identifies individual peaks corresponding to measured ion counts/currents, and assigns identified peaks to specific types of secondary ions 118a based on their respective m/z values.

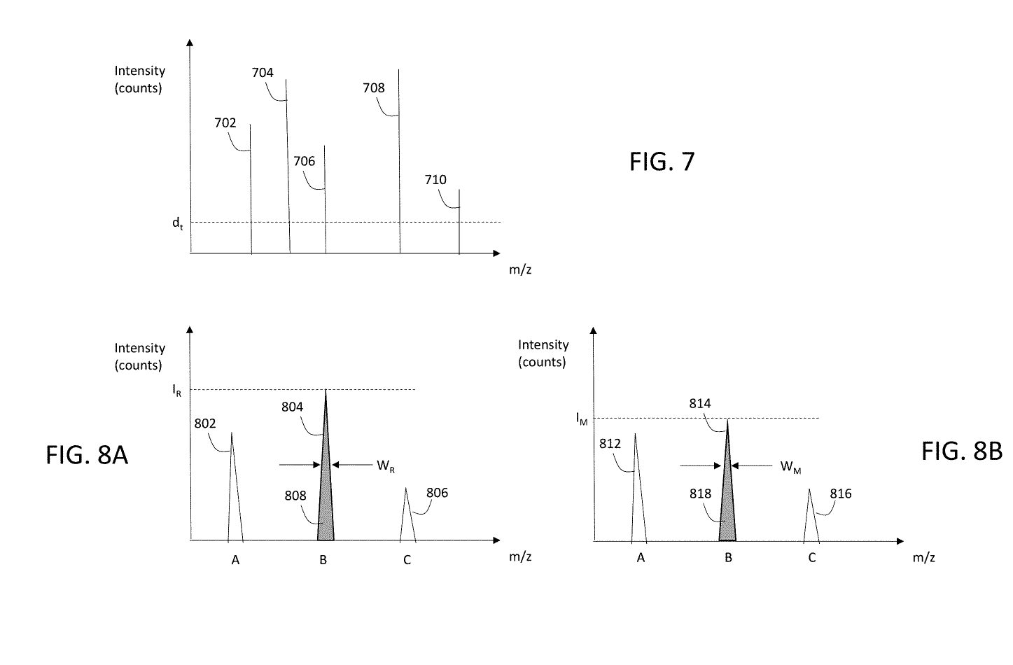

FIG. 7 is a schematic diagram showing representative ion counts/currents measured as a function of m/z by detection apparatus 112 and transmitted to controller 114. Controller 114 analyzes the measured ion counts/currents to identify signal peaks. A variety of peak identification methods can be used by controller 114. For example, in some embodiments, controller 114 identifies peaks in the measured signals by comparing the measured intensity at each m/z value to a signal discrimination threshold value dₜ. At m/z values for which the measured ion count/current exceeds dₜ, controller 114 determines that a signal peak exists.

Referring to FIG. 7, controller 114 identifies five peaks, 702, 704, 706, 708, and 710 among the measured ion counts/currents. After identification, controller 114 compares the m/z values associated with each of the identified peaks to stored reference information to determine secondary ion species associated with each of the identified peaks. As discussed above, the identified peaks may be assigned by controller 114 to correspond to different types of lanthanide ions, to ions generated from coating 154, and to ions generated from substrate 152.

It is also possible that in certain embodiments, one or more identified peaks is not positively correlated with a known type of ion by controller 114. For example, sample 150 can include impurities that generate secondary ion peaks having unrecognized m/z values. Secondary ions can also be generated from the organic components of sample 150. In certain embodiments, ion counts/currents corresponding to such secondary ions are measured by detection apparatus 112. Typically, however, m/z values associated with such ions differ significantly from the expected range of m/z values corresponding to secondary ions generated from mass tags, from coating 154, and from substrate 152. Accordingly, by constraining the range of m/z values over which ion counts/currents are measured in detection apparatus 112, and/or by filtering measured ion counts/currents according to m/z value, secondary ions derived from organic components of sample 150 can generally be excluded from either detection or analysis.

Returning to FIG. 6, in the next step 606, controller 114 obtains a reference distribution of charged particles. In general, the reference distribution of charged particles corresponds to a set of peaks that are expected to be measured when sample 150 is exposed to primary ion beam 116. The reference distribution can be obtained, for example, by retrieving stored calibration information from a storage unit connected to, or embedded within, controller 114. The reference distribution can also be obtained through direct measurement of calibration information by exposing a known portion of sample 150 (or another labeled sample) to primary ion beam 116, and measuring ion counts/currents associated with each of the different types of secondary ions that are generated as a result of the exposure.

As discussed above, the reference distribution of charged particles generally includes a set of peaks and associated m/z values (or a set of associated values of a quantity that is related to m/z) that correspond to known or expected secondary ions generated from a labeled sample. For example, where antibody-conjugated lanthanide elements are used to label sample 150, the reference distribution includes m/z values corresponding to each of the secondary lanthanide ions.

In certain embodiments, the reference distribution also includes intensities (or peak amplitudes) for some or all of the set of m/z values. The amplitudes correspond to expected quantities of each of the different mass tags at the location of incidence of primary ion beam 116. Because the extent of sample tagging varies from one location to another based on biochemical differences between different sample locations, variations in amplitudes of peaks corresponding to each different type of mass tag are generally expected from one location to another within the sample. Accordingly, reference amplitudes within the reference distribution for each type of secondary ion may not always match peak amplitudes for measured secondary ion signals. However, reference amplitudes can be used by controller 114 to perform peak discrimination (e.g., to determine whether a particular peak exists) operations, for example.

In some embodiments, the reference distribution includes peaks corresponding to the set of m/z values. That is, for each m/z value, the reference distribution includes a corresponding peak associated with the m/z value. Controller 114 can analyze the reference peaks to determine properties of the peaks such as peak amplitude, peak area (i.e., area under the peak), and peak width (e.g., a full width at half-maximum amplitude). By combining additional reference information with one or more measured properties of the peaks, controller 114 can also determine parameter values such as a charged particle count associated with each peak.

The reference distribution can also include information about m/z values for peaks corresponding to secondary ions that are not expected to be observed when sample 150 is exposed to primary ion beam 116. For example, the reference distribution can include m/z values associated with secondary ions derived from certain mass tags that have not been applied to sample 150. Given the wide variety of mass tags that can be used in the methods disclosed herein, not all such mass tags may be applied to a given sample 150. Nonetheless, because the reference distribution can be used for samples that are tagged with different combinations of mass tags, the reference distribution can include m/z values for mass tags that are not applied to a specific sample 150.

In addition, the reference distribution can include information about m/z values for types of secondary ions that do not correspond to mass tags. As discussed above, when primary ion beam 116 is incident on coating 154 or substrate 152 instead of sample 150, secondary ions can be generated from coating 154 and substrate 152. Accordingly, the reference distribution can include m/z values associated with secondary ions generated from various materials that are used to form coating 154, and from various materials of substrate 152.

Next, in step 608, controller 114 determines whether one or more deviations exist between the measured secondary ion peaks and the reference distribution of peaks. Identification of deviations by controller 114 determines whether controller 114 implements corrective operations by adjusting the operating parameters of system 100. In general, controller 114 determines whether one or more deviations exist by performing a direct comparison between the set of measured secondary ion peaks from step 604 and the reference distribution obtained in step 606. During the comparison, apparent discrepancies are evaluated by controller 114 to determine whether any of the discrepancies correspond to deviations that cause controller 114 to initiate corrective operations.

A variety of different deviations identified by controller 114 can lead to corrective operations. In some embodiments, controller 114 identifies deviations based on discrepancies between one or more parameters of peaks in the secondary ion signals measured by detection apparatus 112 and peaks in the reference information obtained in step 606. As explained above, controller 114 is configured to determine information corresponding to various parameters of identified peaks in the both the measured secondary ion signals and the reference information. Such parameters include, but are not limited to, peak amplitudes, peak areas (i.e., areas under peaks), peak widths (e.g., full widths at half maximum amplitudes), and charged particle counts associated with particular peaks.

Deviations can be identified by controller 114 when any one or more of the above parameters for corresponding peaks in the measured secondary ion signals and the reference information differ by more than a threshold amount. FIG. 8A is a schematic diagram showing measured secondary ion signals for a sample 150 as a function of m/z. In FIG. 8A, three peaks 802, 804, and 806 are present at m/z values of A, B, and C, respectively. FIG. 8B is a schematic diagram showing a reference distribution of charged particle peaks for sample 150. In FIG. 8B, three peaks 812, 814, and 816 are present at m/z values of A, B, and C, respectively.

To identify deviations between the measured secondary ion signals for sample 150 and the reference distribution, controller 114 can determine information from each of the signals shown in FIGS. 8A and 8B. As a first step, for example, controller 114 identifies each of the peaks 802, 804, 806, 812, 814, and 816, and the m/z values (A, B, C) associated with each peak. For each peak 802, 804, and 806 present in the measured secondary ion signals, corresponding peaks 812, 814, and 816 are present at the same m/z values in the reference distribution. Accordingly, no differences exist in the m/z values of the identified peaks.

Controller 114 can then analyze each of the peaks to determine quantitative information for the peaks, and compare the quantitative information for the measured secondary ion signals and reference distribution to identify deviations. For example, with reference to corresponding peaks 804 and 814, controller 114 can determine the peak amplitude of each peak. In FIGS. 8A and 8B, peak 804 has an amplitude I_{R} while peak 814 has an amplitude I_{M}. Controller 114 can determine that a deviation exists between the measured secondary ion signals and the reference distribution if the difference between amplitudes I_{R} and I_{M} exceeds a threshold value for the amplitude difference between peaks 804 and 814.

Controller 114 can also determine the areas under each of peaks 804 and 814 in FIGS. 8A and 8B, which are labeled 808 and 818 respectively. Controller 114 can determine that a deviation exists between the measured secondary ion signals and the reference distribution if the difference between areas 808 and 818 exceeds a threshold value for the difference in areas under peaks 804 and 814.

Controller 114 can further determine the widths (e.g., the full widths at half-maximum amplitude) of each of peaks 804 and 814 in FIGS. 8A and 8B. As shown in the figures, peak 804 has a width W_{R} and peak 814 has a width W_{M}. Controller 114 can then determine that a deviation exists between the measured secondary ion signals and the reference distribution if the difference between widths W_{R} and W_{M} exceeds a threshold value for the difference in widths between peaks 804 and 814.

As discussed above, controller 114 can also combine additional reference information with measured parameters associated with identified peaks to determine additional information for the peaks. For example, based on at least one of an area under the peaks, an amplitude of the peaks, and a width of the peaks, controller 114 can determine charged particle counts represented by each of the identified peaks. Referring to FIGS. 8A and 8B for example, controller 114 can determine charged particle counts (i.e., secondary ion counts) associated with each of peaks 804 and 814. If a difference in the charged particle counts associated with peaks 804 and 814 is larger than a threshold value for the difference in charged particle counts, controller 114 determines that a deviation exists between the measured secondary ion signals and the reference distribution.

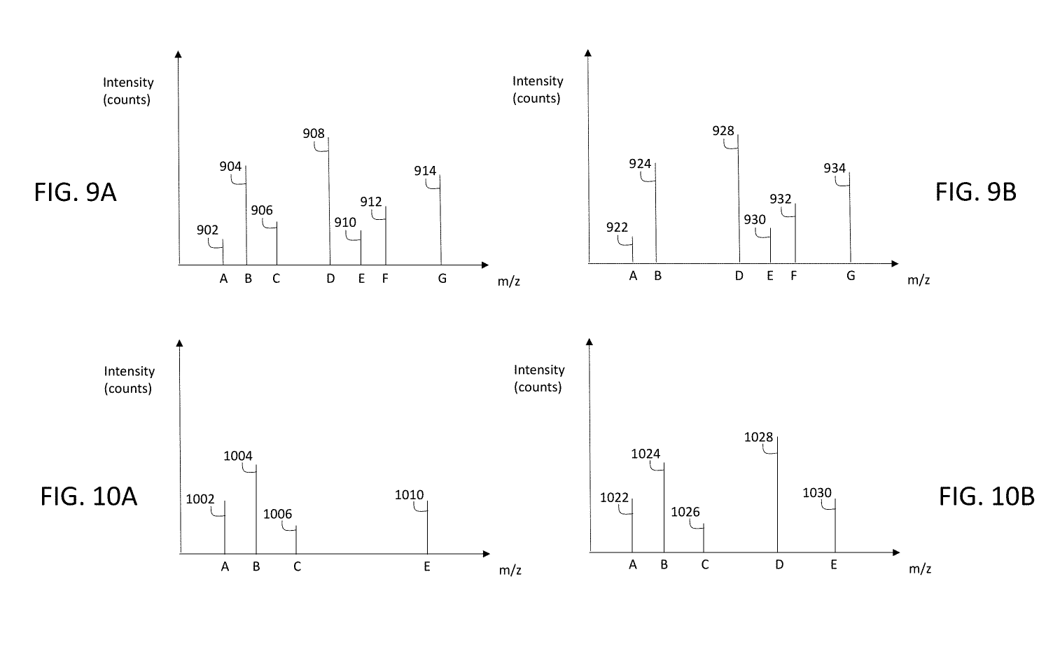

In some embodiments, deviations between the measured secondary ion signals and the reference distribution are identified by controller 114 based on the presence or absence of certain peaks in the measured secondary ion signals. For example, controller 114 can determine that a deviation exists when one or more peaks that are identified in the reference distribution are not identified in the measured secondary ion signals. FIG. 9A is a schematic diagram of a reference distribution of peaks for a sample. The reference distribution includes peaks 902, 904, 906, 908, 910, 912, and 914 at m/z values A, B, C, D, E, F, and G, respectively. FIG. 9B is a schematic diagram showing measured secondary ion signals for the sample. The measured signals include peaks 922, 924, 928, 930, 932, and 934 at m/z values A, B, D, E, F, and G.

By comparing the reference distribution in FIG. 9A and the measured secondary ion signals in FIG. 9B, controller 114 identifies that peak 906 at a m/z value of C in the reference distribution is not present in the measured secondary ion signals. Accordingly, controller 114 determines that a deviation exists between the measured secondary ion signals and the reference distribution.

The deviation identified by comparing FIGS. 9A and 9B can arise in various ways. In certain embodiments, for example, the peak that is missing in the measured secondary ion signals corresponds to secondary ions generated from an organic portion of sample 150. When primary ion beam 116 is incident on sample 150, the reference distribution indicates that at least some secondary ions are expected to be generated from the sample. The absence of a measured peak corresponding to such secondary ions indicates that primary ion beam 116 may not be incident on sample 150 (and, for example, may instead be incident on coating 154 or substrate 152).

In certain embodiments, controller 114 determines that a deviation exists between the measured secondary ion signals and the reference distribution if, for one or more peaks in the reference distribution, corresponding peaks in the measured secondary ion signals have values of peak properties that are less than threshold values from the reference distribution. For example, rather than the complete absence of a peak corresponding to reference distribution peak 906 in the measured secondary ion signals as shown in FIG. 9B, controller 114 may instead identify a corresponding peak at a m/z value of C in the measured secondary ion signals. However, parameter values associated with the identified peak may be less than threshold values of the parameters from the reference distribution. For example, a peak amplitude of the identified peak at a m/z value of C in the measured secondary ion signals may be less than a threshold value of peak amplitude for peak 906 in the reference distribution. Alternatively, or in addition, an area under the identified peak at a m/z value of C in the measured secondary ion signals may be less than a threshold value of the area under peak 906 in the reference distribution. In these circumstances, even though a peak is identified at a m/z value of C in the measured secondary ion signals, controller 114 still determines that a deviation exists between the measured secondary ion signals and the reference distribution.

In some embodiments, an opposite situation from the one shown in FIG. 9A and FIG. 9B occurs, and one or more peaks are identified in the measured secondary ion signals that do not have counterparts in the reference distribution. FIG. 10A is a schematic diagram showing a reference distribution for a sample that includes peaks 1002, 1004, 1006, and 1010 at m/z values of A, B, C, and E, respectively. FIG. 10B is a schematic diagram showing measured secondary ion signals for the sample, and includes identified peaks 1022, 1024, 1026, 1028, and 1030 at m/z values of A, B, C, D, and E, respectively. As is apparent, controller 114 identifies peak 1028 in the measured secondary ion signals as having no counterpart in the reference distribution, and therefore a deviation exists between measured secondary ion signals and the reference information.

The situation shown in FIGS. 10A and 10B can arise, for example, when peak 1028 corresponds to secondary ions that are not derived from mass tags or an organic component of the sample. If primary ion beam 116 is not incident on the sample but is instead incident on a portion of coating 154 (e.g., because the portion of sample 150 previously at the location of incidence 124 of primary ion beam 116 has been ablated by the primary ion beam, or because the location of incidence corresponds to a hole in sample 150), then peak 1028 can correspond to secondary ions generated from coating 154 (e.g., gold ions, tantalum ions, titanium ions, chromium ions, tin ions, and/or indium ions). By determining the identity of the secondary ions corresponding to peak 1028 based on the m/z value for the peak, controller 114 determines that primary ion beam 116 is no longer incidence on sample 150.

As another example, the situation shown in FIGS. 10A and 10B can arise, when primary ion beam 116 is incident on a portion of substrate 152 (e.g., because the portion of sample 150 previously at the location of incidence 124 of primary ion beam 116 has been ablated by the primary ion beam as has any coating 154 at the location of incidence 124, or because the location of incidence corresponds to a hole in sample 150 and any coating 154 underlying the hole has been ablated). In these circumstances, peak 1028 can correspond to secondary ions generated from substrate 152 (e.g., silicon ions). As above, by determining the identity of the secondary ions corresponding to peak 1028 based on the m/z value for the peak, controller 114 determines that primary ion beam 116 is no longer incidence on sample 150.

In certain embodiments, controller 114 is configured to identify a deviation between the measured secondary ion signals and the reference distribution when one or more peaks that are present in measured secondary ion signals but not in the reference distribution have associated peak parameter values that exceed threshold values of the reference distribution. For example, referring to FIGS. 10A and 10B, controller 114 determines that a deviation exists when a peak amplitude of peak 1028 exceeds a reference distribution threshold for peak amplitude, and/or when a peak width of peak 1028 exceeds a reference distribution threshold for peak width, and/or when an area under peak 1028 exceeds a reference distribution value for the peak area. In effect, identifying deviations in this fashion is equivalent to applying at least two criteria for peak 1028: peak 1028 satisfies at least one peak identification criterion so that controller 114 identifies peak 1028, and peak 1028 also satisfies at least one peak parameter criterion whereby controller 114 determines that a peak parameter value for peak 1028 exceeds a corresponding threshold value from the reference distribution.

Returning to FIG. 6, after controller 114 has determined that a deviation exists between the measured secondary ion signals and the reference distribution in step 608, controller 114 adjusts at least one exposure parameter of primary ion beam 116 in response to the identified deviation in step 610. In general, the adjustments to the exposure parameter are performed to ensure that primary ion beam 116 does not generate secondary ions from regions other than those that correspond to sample 150.

Where controller 114 determines that primary ion beam 116 is not incident on a portion of sample 150 (e.g., because the measured secondary ion signals include one or more peaks corresponding to secondary ions generated from coating 154 and/or substrate 152, and/or because the measured secondary ion signals include one or more peaks corresponding to secondary ions generated from the sample that have peak parameter values below corresponding threshold values from the reference distribution), controller 114 can implement a variety of adjustments to the exposure parameters. In some embodiments, controller 114 terminates exposure at the location of incidence 124 to the primary ion beam 116.

Terminating exposure to primary ion beam 116 can include, for example, directing primary ion beam 116 to a new location of incidence 124 by adjusting components of ion beam optics 104 and/or by translating stage 106, as discussed above. Alternatively, controller 114 can terminate exposure at the location of incidence 124 to primary ion beam 116 by directing primary ion beam 116 to be incident on a beam blocking element (e.g., beam blocking elements 512 in ion beam optics 104). As discussed above, controller 114 can direct primary ion beam 116 to be incident on beam blocking elements 512 by adjusting electrical potentials applied to deflecting elements in ion beam optics 104.

In certain embodiments, controller 114 reduces an ion current of primary ion beam 116 when a deviation is identified to limit the dose of primary ions 116a delivered at the location of incidence 124 by primary ion beam 116. Controller 114 adjusts the ion current of primary ion beam 116 by transmitting a suitable control signal to ion source 102, as discussed above. In general, the amount by which the ion current is reduced can be selected as desired to limit the dose of primary ions 116a. In some embodiments, for example, when controller 114 determines that a deviation exists, controller 114 reduces the ion current of primary ion beam 116 by 50% or more (e.g., 70% or more, 80% or more, 90% or more, 95% or more, 98% or more, 99% or more, 99.9% or more, 99.999% or more).

In some embodiments, controller 114 de-activates ion source 102 when a deviation is identified, via a suitable control signal transmitted to ion source 102. De-activating ion source 102 can have certain advantages, depending upon the nature of ion source 102. For example, certain ion sources are limited in average output power; by de-activating such a source when a deviation is detected, larger ion beam output powers can be realized when ion source 102 is re-activated by controller 114.

As discussed above, the exposure pattern 400 is maintained by controller 114, and in some embodiments, sample 150 is exposed to primary ion beam 116 multiple times according to exposure pattern 400. Accordingly, when controller 114 identifies that a deviation exists between the measured secondary ion signals and the reference distribution for a particular exposure region, controller 114 can adjust one or more exposure parameters for primary ion beam 116 for a subsequent exposure of the region to the primary ion beam. For example, as discussed above, for each region in exposure pattern 400, the exposure pattern includes an associated exposure time or dwell time which represents the period of time during which the region is exposed to primary ion beam 116. In certain embodiments, when controller 114 determines that a deviation exists for a region in exposure pattern 400, controller 114 reduces a dwell time of the primary ion beam 116 for the region in a subsequent exposure of the region to primary ion beam 116 (e.g., when the region is revisited again by primary ion beam 116 upon a subsequent execution of exposure pattern 400). The dwell time for the region can be reduced by 50% or more (e.g., 70% or more, 80% or more, 90% or more, 95% or more, 98% or more, 99% or more, 99.9% or more, 99.999% or more) for a subsequent exposure of the region.

Alternatively, or in addition, when controller 114 determines that a deviation exists for a region in exposure pattern 400, controller 114 reduces an ion current of primary ion beam 116 in a subsequent exposure of the region to primary ion beam 116. The primary ion beam current can be reduced by 50% or more (e.g., 70% or more, 80% or more, 90% or more, 95% or more, 98% or more, 99% or more, 99.9% or more, 99.999% or more) for a subsequent exposure of the region.

As another alternative, when controller 114 determines that a deviation exists for a region in exposure pattern 400, controller 114 can de-activate ion source 102 when the region is revisited during a subsequent execution of exposure pattern 400. As discussed above, de-activation of ion source 102 can have certain advantages for sources that are limited in average output power.

The foregoing adjustments of exposure parameters apply to subsequent exposures of regions of the sample to primary ion beam 116. To implement such adjustments, controller 114 can adjust exposure pattern 400. That is, controller 114 can adjust dwell times, ion beam currents, and other exposure parameters that are associated with specific regions in exposure pattern 400. As discussed above, these exposure parameters are part of exposure pattern 400, and adjustment of these exposure parameters constitutes an adjustment of exposure pattern 400.

In general, when a sample is exposed to primary ion beam 116 multiple times according to an exposure pattern 400, adjusting ion beam exposure parameters for the exposure pattern is more straightforward to implement than adjusting the spatial locations of the exposure pattern. That is, by adjusting ion beam exposure parameters but maintaining the same set of spatial locations in exposure pattern 400 between subsequent exposures of sample 150 to primary ion beam 116, controlling the location of incidence 124 of primary ion beam 116 as it is scanned over sample 150 is simpler.

However, in certain embodiments, when controller 114 determines that a deviation exists between the measured secondary ion signals and the reference distribution, controller 114 adjusts the set of spatial locations associated with exposure pattern 400. In other words, for exposure regions for which controller 114 determines that a deviation exists, controller 114 can adjust exposure pattern 400 by removing spatial locations corresponding to the regions from the set of spatial locations associated with the exposure pattern. As discussed above, FIG. 4A shows one example of an exposure pattern 400 according to which sample 150 is exposed to primary ion beam 116.

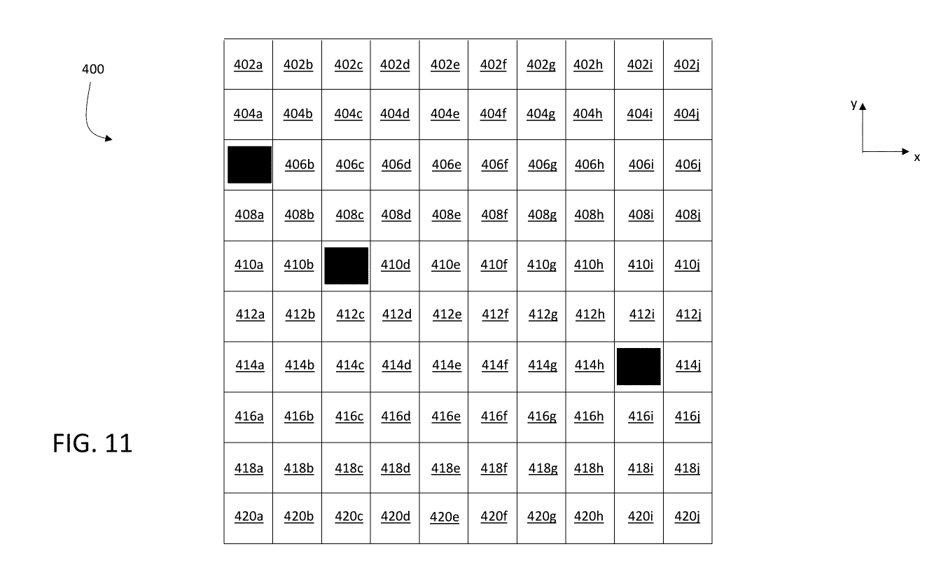

If, for example, controller 114 determines that deviations exist when sample 150 is exposed to primary ion beam 116 at spatial locations 406a, 410c, and 414i, then controller 114 can modify exposure pattern 400 to remove these spatial locations from the exposure pattern. A modified exposure pattern 1102 is shown in FIG. 11, with these spatial locations removed (shown as darkened regions in FIG. 11).

Thus, when sample 150 is exposed to primary ion beam 116 multiple times according to a first exposure pattern that defines a first exposure sequence, controller 114 can modify ion beam exposure parameters and/or spatial locations associated with the first exposure to generate a second, updated exposure pattern defining a different exposure sequence for sample 150 during a subsequent exposure of the sample to primary ion beam 116. When only modifications to the ion beam exposure parameters occur, the second exposure pattern can include the same set of spatial locations as the first exposure pattern. However, when the set of spatial locations is modified by controller 114, the second exposure pattern can include fewer spatial locations than the first exposure pattern.

Returning to FIG. 6, after adjustment of the ion beam exposure parameters is complete in step 610, the procedure terminates at step 612.

In addition to detecting deviations based upon differences between peaks in the measured secondary ion signals and the reference distribution, in some embodiments controller 114 can also implement other criteria to determine whether to adjust ion beam exposure parameters. For example, in certain embodiments, controller 114 can determine, from the measured secondary ion signals, a total ion yield from the sample (e.g., based on areas under the peaks that correspond to secondary ions generated from the sample). The reference distribution can include a threshold value for the total secondary ion yield from the sample, and by comparing the measured total ion yield to the threshold value, controller 114 can determine whether a deviation exists between the measured secondary ion signals and the reference distribution. If controller 114 determines that a deviation exists (i.e., if the total secondary ion yield is less than the threshold value), controller 114 can implement any of the adjustments discussed above.

In certain embodiments, detection apparatus 112 can measure secondary electrons generated from sample 150 when primary ion beam 116 is incident on the sample. Detection apparatus 112 generates an output signal that includes information about the quantity of secondary electrons detected, and the output signal is received by controller 114 via signal line 120f The reference distribution can include a threshold value for the secondary electron yield from the sample, and by comparing the measured secondary electron yield to the threshold value, controller 114 can determine whether a deviation exists between the measured secondary electron yield and the reference distribution. If controller 114 determines that a deviation exists (i.e., if the secondary electron yield is less than the threshold value), controller 114 can implement any of the adjustments discussed above.

### (vi) Predictive Adjustment of Operating Parameters

As discussed in the previous section, controller 114 is configured to implement a variety of adjustments to the operating parameters of system 100 (e.g., ion beam exposure parameters) based on various criteria for identifying deviations between charged particle signals (e.g., secondary ion signals, secondary electron signals) measured by detection apparatus 112 and a reference charged particle distribution. As is evident from the examples discussed, the reference charged particle distribution can include various types of information, including m/z values for peaks corresponding to detected charged particles, peak amplitudes, peak profiles (i.e., electrical signals corresponding to the measured peaks, from which various peak parameters can be determined by controller 114), threshold values, and information about peaks corresponding to other ions that are not expected to be observed at particular locations of incidence 124 of primary ion beam 116.

In the previous section, controller 114 implements adjustments reactively based on an identified deviation. That is, identified deviations act as triggers for the implementation of adjustments by controller 114. In some embodiments, however, controller 114 is configured to determine information about a sample predictively, and to implement adjustments to exposure parameters in prospective fashion. Predictive adjustments can be implemented in addition to, or as an alternative to, adjustments triggered by identified deviations between measured charged particle signals and reference distributions.

In general, predictive information determination and parameter adjustments are performed by controller 114 based on analyses of time-dependent measurements of charged particle signals by detection apparatus 112. Controller 114 determines, from the temporal evolution of such signals, information about the sample at locations of incidence 124 of primary ion beam 116.

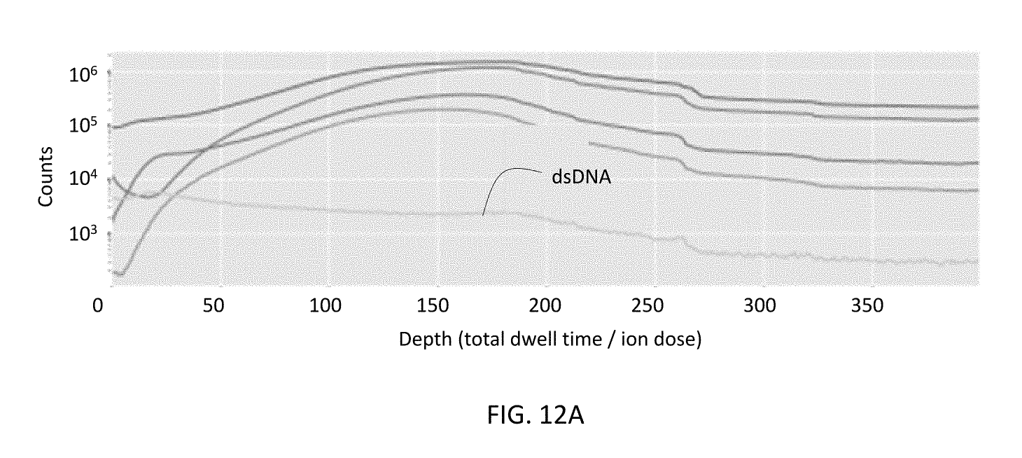

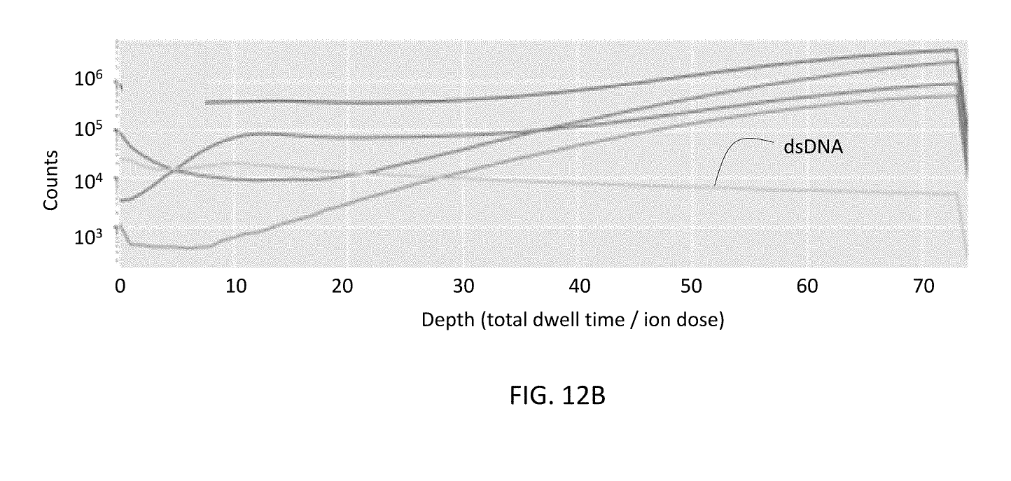

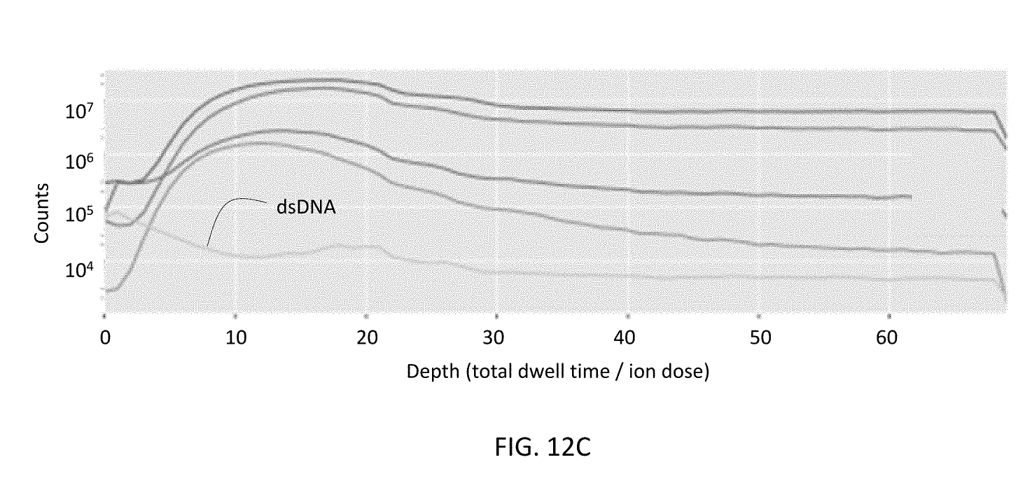

FIGS. 12A-12C show time-dependent measurements of secondary ion signals by detection apparatus 112 for several different types of secondary ions. The measurement units are detected ion counts as a function of depth, where depth is calculated as a ratio of total dwell time at the location of incidence 124 of primary ion beam 116 to a total ion dose delivered by primary ion beam 116 to the location of incidence 124. The data shown in FIGS. 12A-12C are integrated over all exposed regions of the sample. The labeled curves correspond to the following detected secondary ions: “Na” corresponds to detected sodium ions, which are typically generated from impurities; “Si” corresponds to detected silicon ions, which are typically generated from substrate 152; “Ta” corresponds to detected tantalum ions and “Au” corresponds to detected gold ions, which are each typically generated from coating 154; and “dsDNA” corresponds to ions generated from a mass tag used to label the sample.

FIG. 12A corresponds to a dwell time of 0.25 ms for primary ion beam 116 on each exposed region of the sample, while FIGS. 12B and 12C correspond to dwell times of 1 ms and 4 ms on each exposed region of the sample. As is evident from the figures, the detected ion counts vary over time (note that depth is related to time along the horizontal axis of each plot). In particular, ion counts for the dsDNA mass tag generally decline as portions of the sample are ablated by the primary ion beam, exposing coating 154 and the, substrate 152 to the primary ion beam. As coating 154 and substrate 152 are exposed, ion counts associated with secondary ions generated from the coating and substrate rise. Ion counts associated with coating 154 eventually decline as coating 154 is ablated by the primary ion beam. In contrast, ion counts associated with substrate 152 generally reach an approximate plateau, as the primary ion beam never ablates all the way through substrate 152.

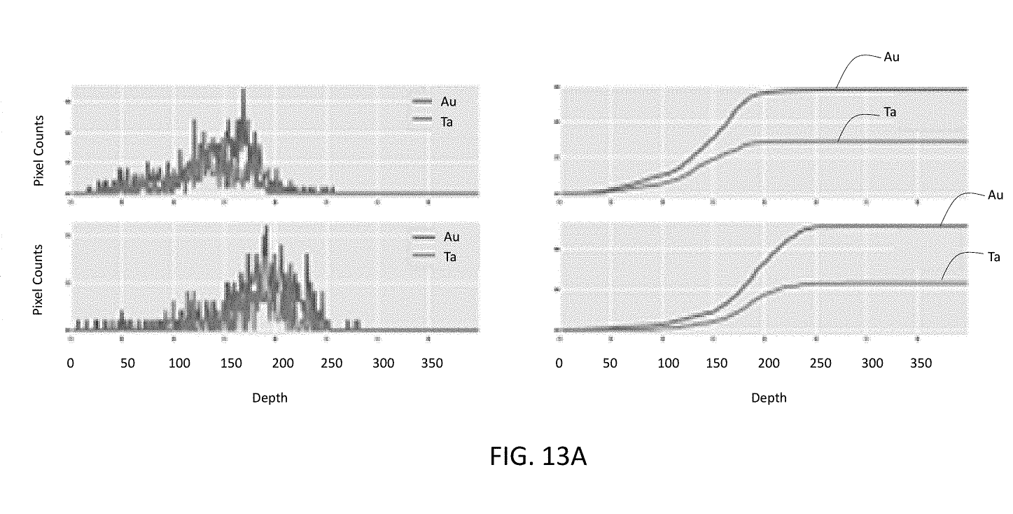

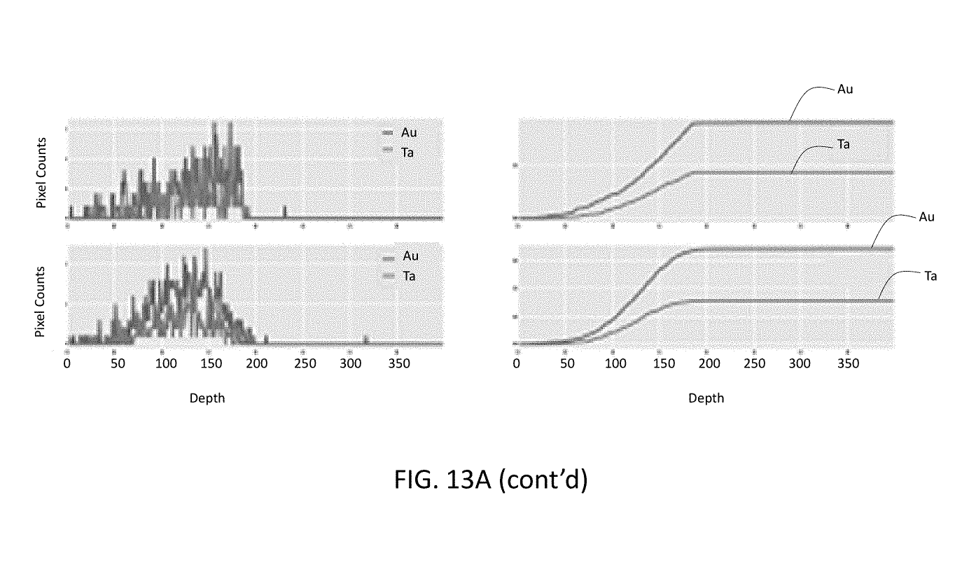

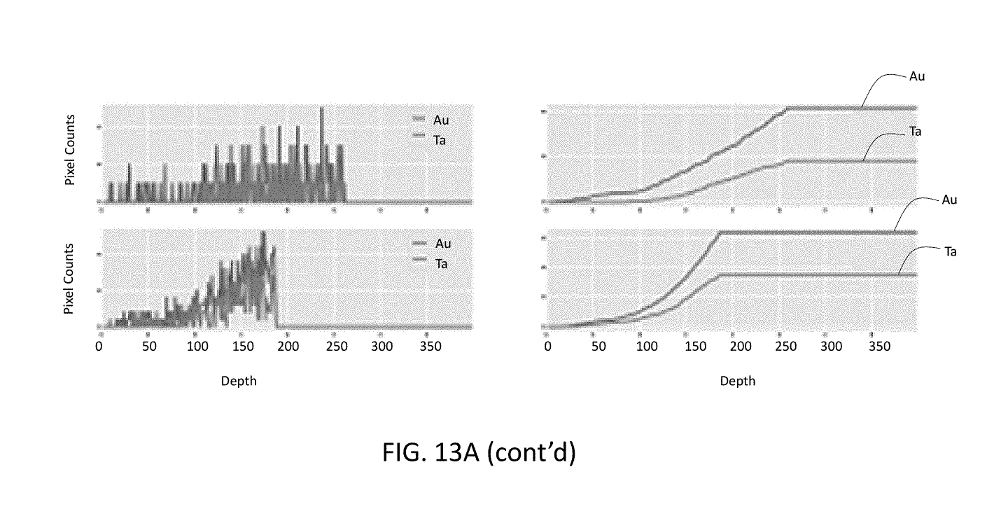

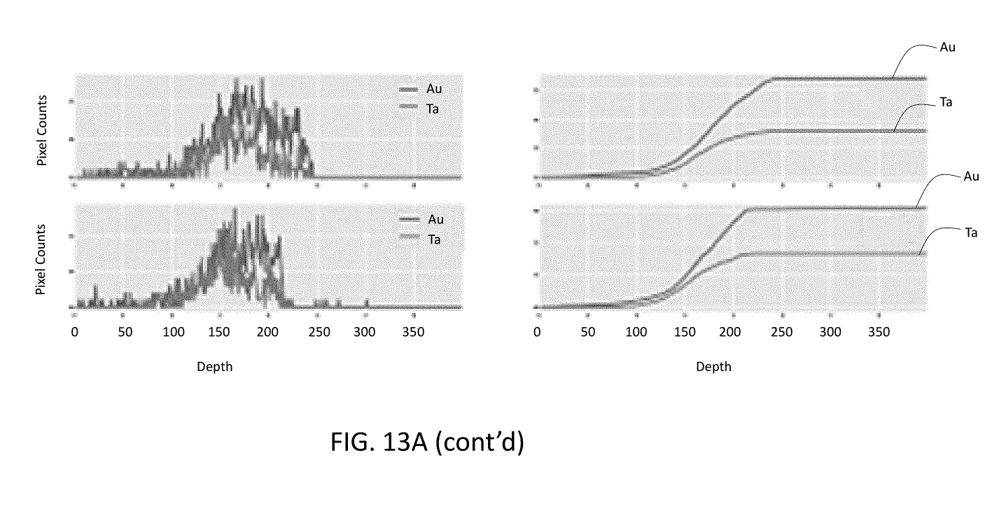

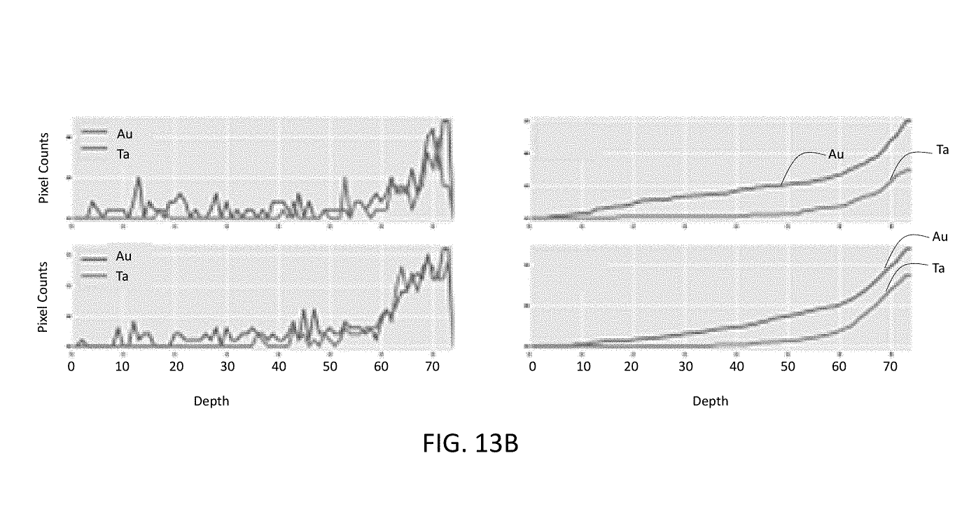

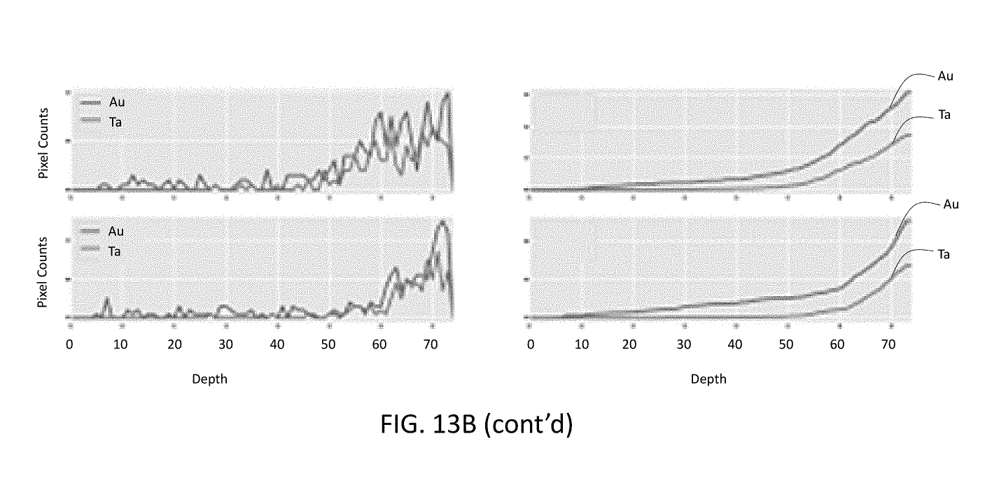

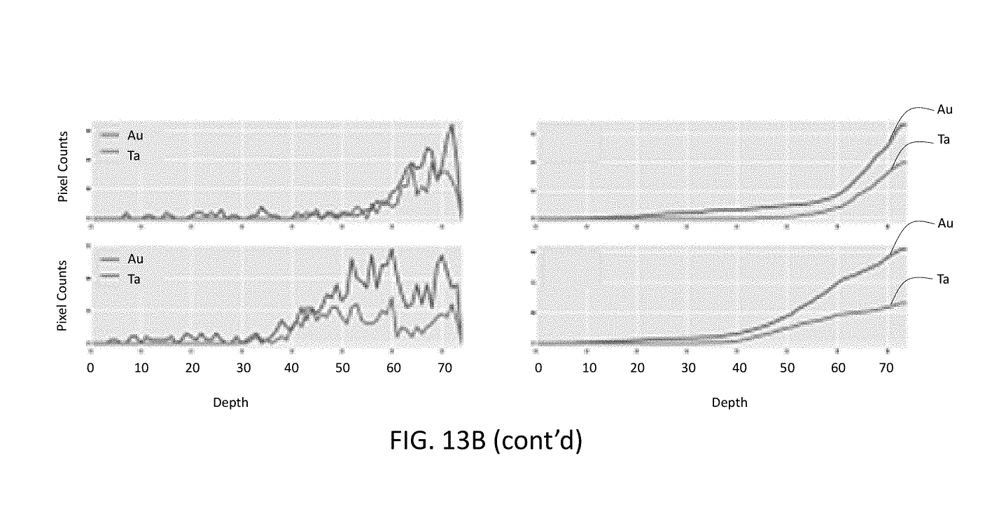

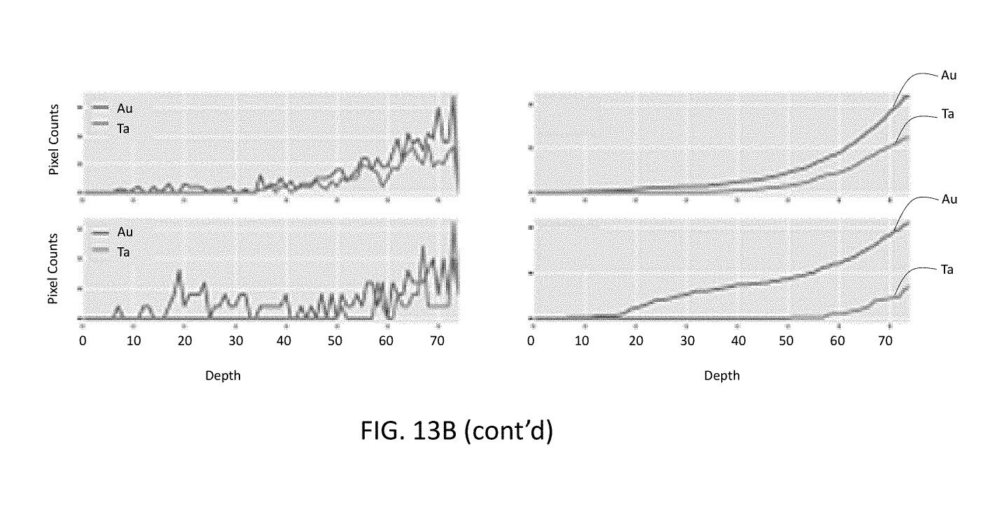

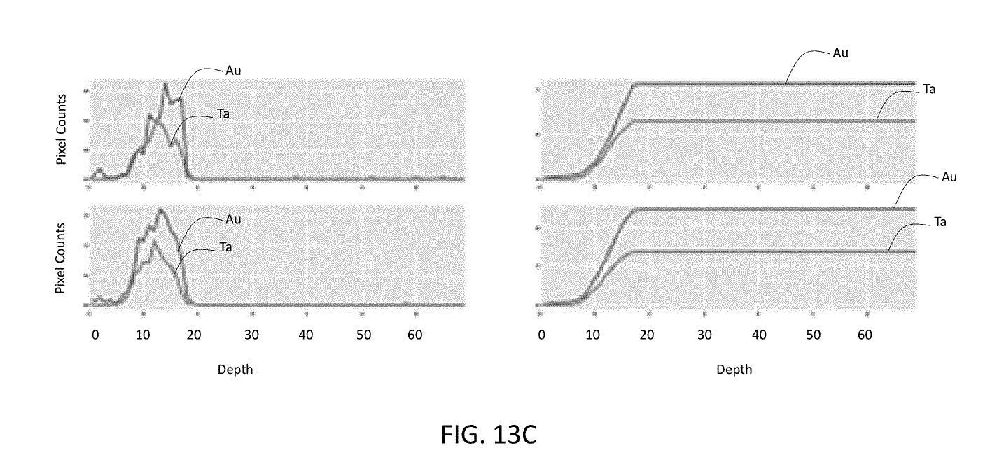

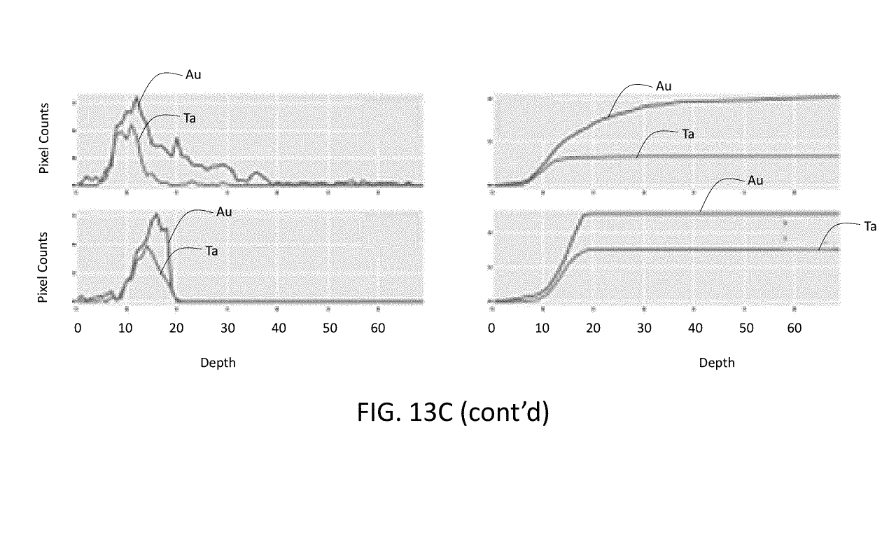

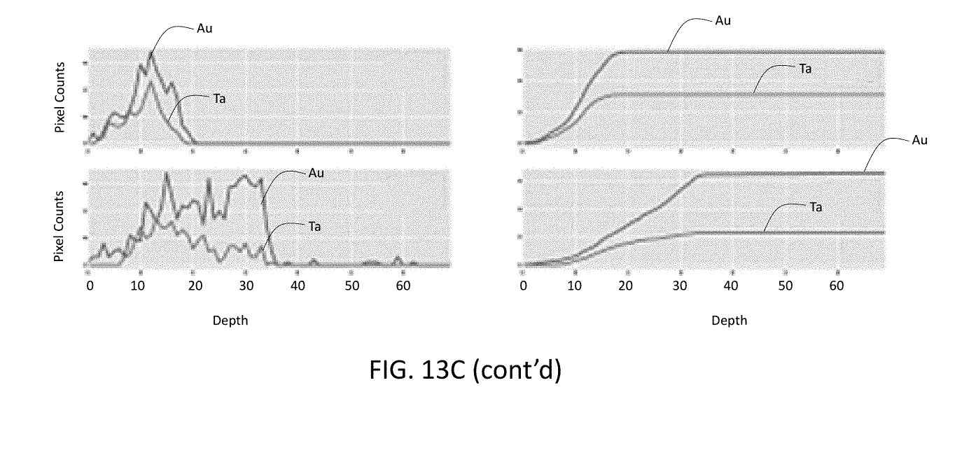

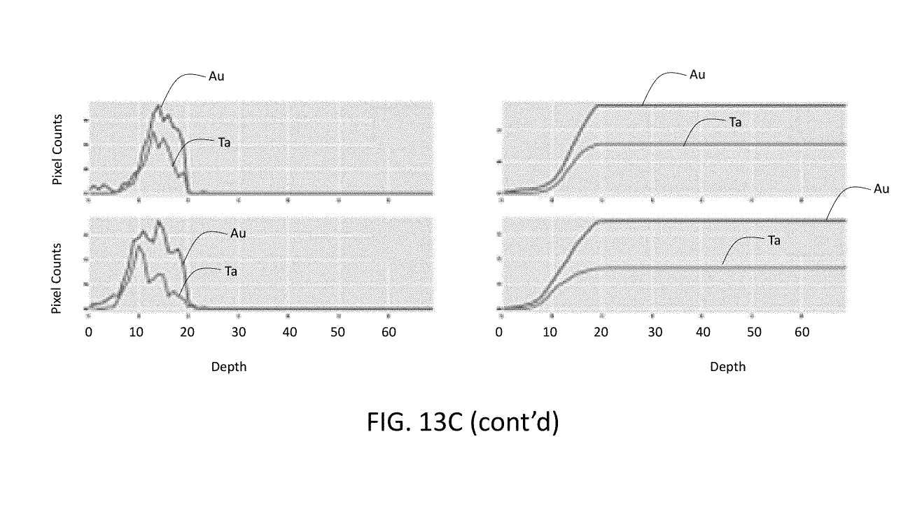

Because of inhomogeneity in sample 150 (e.g., regions of different tissue density within the sample, as well as holes and other non-uniform features), ion counts associated with secondary ions generated from coating 154 and substrate 152 increase at different times for different spatial locations within the sample that are exposed to the primary ion beam. FIG. 13A shows a set of plots of instantaneous measured ion counts (left hand plots in the figure) and cumulative measured ion counts (right hand plots in the figure) for 8 different spatial locations within a sample exposed to the primary ion beam. The dwell time at each location was 0.25 ms. The spatial locations are labeled “Pixel 12170”, “Pixel 59995”, “Pixel 49330”, “Pixel 51993”, “Pixel 46221”, “Pixel 28202”, “Pixel 8873”, and “Pixel 44296” in FIG. 13A.

Similarly, FIGS. 13B and 13C each show a set of plots of instantaneous measured ion counts (left hand plots in the respective figures) and cumulative measured ion counts (right hand plots in the respective figures) for 8 different spatial locations within a sample exposed to the primary ion beam. The dwell time at each location was 1 ms in FIG. 13B and 4 ms in FIG. 13C. The noise level in the measured ion counts decreases as the dwell time increases.

As shown in FIGS. 13A-13B, measured instantaneous ion counts for Au and Ta ions (which are generated from coating 154) increase at a relatively rapid rate initially, plateau briefly, and then decrease relatively rapidly again. The cumulative ion counts show an initial period during which Au and Ta ions are detected in increasing numbers, and then a longer period after the decay of the instantaneous ion counts when few additional Au and Ta ions are detected. The observed data are consistent with the assumption that initially, as the primary ion beam ablates the remaining portion of sample 150 at each location of incidence 124, secondary Au and Ta ions begin to be generated from coating 154. As the primary ion beam ablates the final portion of coating 154 at each location of incidence 124, generation of secondary Au and Ta ions declines.

In the following examples, thresholds based on measured secondary Au and Ta ions therefore function as indicators that at each location of incidence 124, primary ion beam has ablated sample 150 and is incident on the underlying coating 154. However, it should be appreciated that thresholds based on measured secondary ion counts for any of the different types of secondary ions discussed herein can be used to determine suitable thresholds, as the rates of generation of each of the different types of ions show variations when primary ion beam 116 reaches the interface between sample 150 and coating 154 or substrate 152.

In some embodiments, for exposure of a region of sample 150 to primary ion beam 116, controller 114 can determine that the primary ion beam has reached coating 154 (e.g., by ablating through sample 150, or when the location of incidence 124 corresponds to a naturally occurring hole in sample 150) based on a simple threshold condition. For example, controller 114 can determine that coating 154 has been reached by primary ion beam 116 if the instantaneous count of secondary Au or Ta ions exceeds a certain threshold ion count value. In some circumstances, however, simple threshold conditions can be prone to the effects of outliers among the measured instantaneous ion counts. For example, a single anomalously high instantaneous ion count that is larger than the threshold value can trigger additional operations by controller 114 under the assumption that the primary ion beam has reached coating 154.

Accordingly, in certain embodiments, controller 114 is configured to determine appropriate threshold values using alternative methods. For example, controller 114 can be configured to determine threshold values using changepoint analysis. Suitable methods for determining changepoints and their associated confidence levels for time-series data are described, for example, in Killick et al., “changepoint: An R Package for Changepoint Analysis,” J. Stat. Software 58(3): 1-19 (2014), and in Killick et al., “Optimal Detection of Changepoints with a Linear Computational Cost,” J. Am. Stat. Assoc. 107(500): 1590-1598 (2012), the entire contents of each of which are incorporated herein by reference.

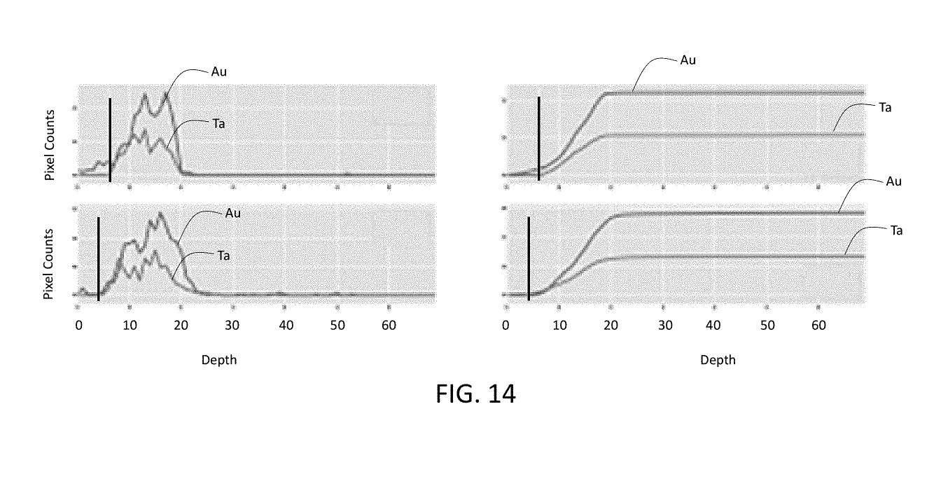

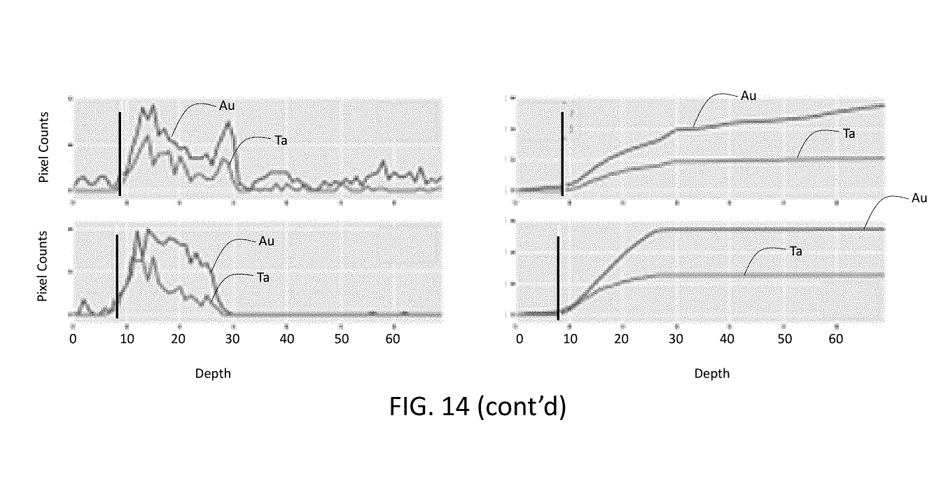

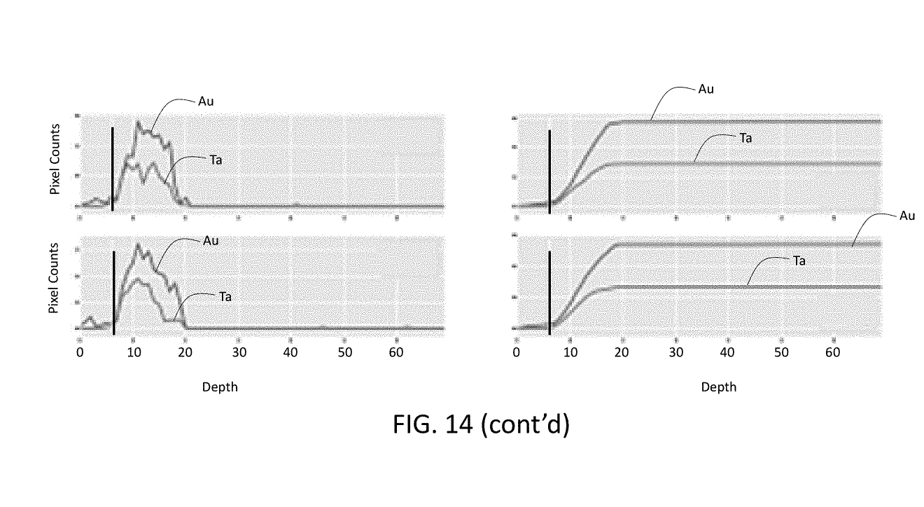

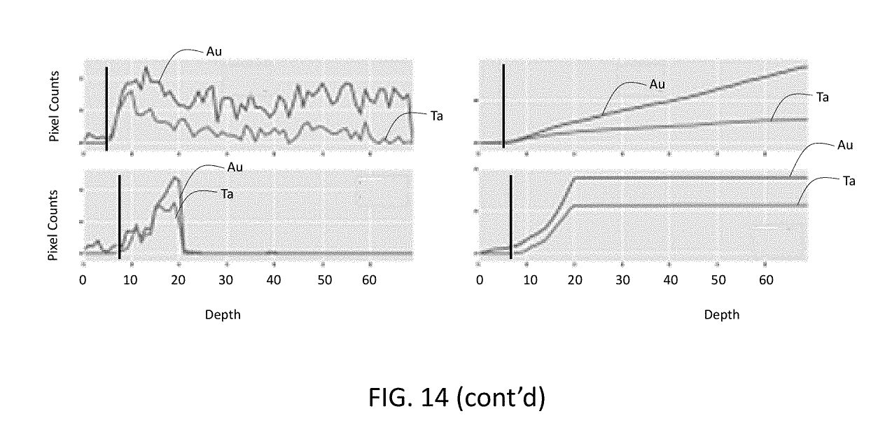

FIG. 14 shows a set of plots of instantaneous measured ion counts (left hand plots in the figure) and cumulative measured ion counts (right hand plots in the figure) for 8 different spatial locations within a sample exposed to the primary ion beam. The first changepoints determined for each of the instantaneous plots and each of the cumulative plots are shown as vertical lines. These changepoints can function as threshold values and can be used by controller 114 to determine when primary ion beam 116 has reached coating 154.

While individual thresholds can be determined based on changepoints for each sample location that is exposed to primary ion beam 116, it can be more efficient to determine changepoints based on multiple different locations of incidence 124 within a sample. “Universal” changepoints determined in this manner can function as universal instantaneous and cumulative threshold values for all locations of incidence 124.

In general, to determine suitable universal thresholds for all sample locations, calibration information is first measured for a calibration sample. Measurement of calibration information involves measurement of instantaneous and/or cumulative secondary ion counts at multiple locations of incidence 124 of primary ion beam 116 in the calibration sample, and at each exposed region of the sample, determining instantaneous and cumulative changepoint values. Controller 114 can then determine suitable universal changepoint values for use as thresholds indicating that primary ion beam 116 has reached coating 154 from the distributions of instantaneous and cumulative changepoint values for the calibration sample.

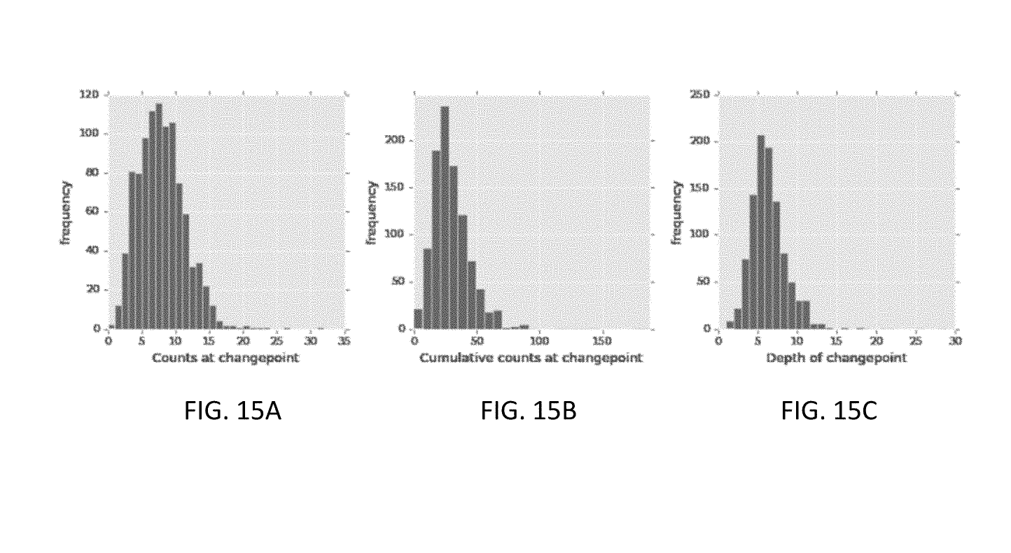

FIGS. 15A-15C are histograms showing instantaneous Au ion counts at the first changepoint (FIG. 15A), cumulative Au ion counts at the first changepoint (FIG. 15B), and depth values at the first changepoint (FIG. 15C) for measured signals from a calibration sample. Based on the distributions of changepoints shown in FIGS. 15A and 15B, controller 114 can determine suitable universal instantaneous and cumulative thresholds based on the distributions of instantaneous and cumulative changepoints, respectively. For example, in some embodiments, controller 114 can determine a universal instantaneous threshold as the mean, median, or mode of the distribution of instantaneous changepoint values shown in FIG. 15A. Similarly, controller 114 can determine a cumulative threshold as the mean, median, or mode of the distribution of cumulative changepoint values shown in FIG. 15B. More generally, suitable universal threshold values can also be determined by controller 114 based on more complex metrics derived from the distributions shown in FIGS. 15A and 15B.

The universal threshold values determined by controller 114 are used by the controller to exclude from measured data secondary ion signals arising after the universal threshold values are first exceeded at each location of incidence 124, under the assumption that the excluded signals arise from secondary ions generated in coating 154 after sample 150 has been fully ablated at the location of incidence. This process of measured data exclusion amounts essentially to filtering the measured signals that are used to construct images of sample 150.

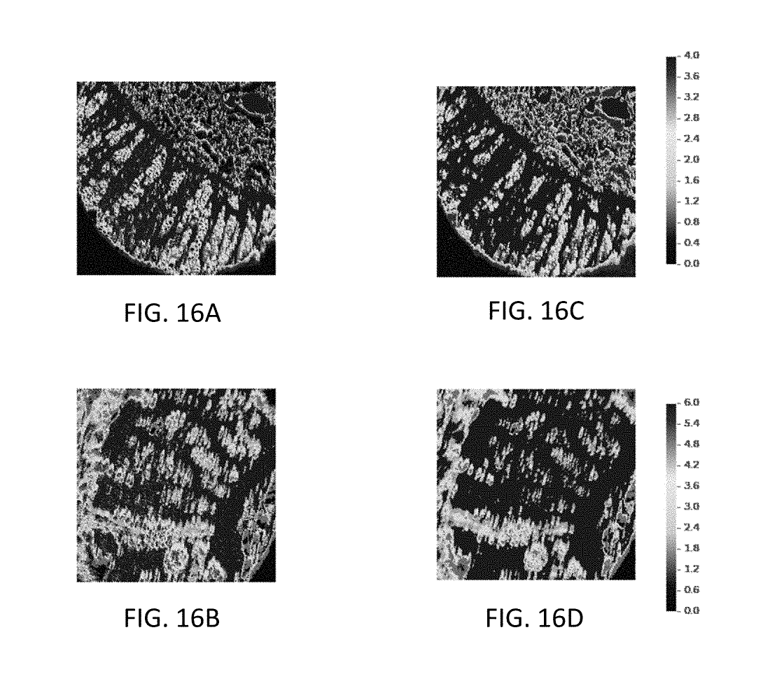

In general, both universal instantaneous threshold values and universal cumulative threshold values can separately be used to effectively exclude certain measured secondary ion signals. FIGS. 16A and 16B show images of two different samples, each constructed from measured secondary ion signals filtered according to a universal instantaneous threshold value determined as discussed above. FIGS. 16C and 16D show images of the same two samples, respectively, each constructed from measured secondary ion signals filtered according to a universal cumulative threshold value determined as discussed above. Corresponding images of each sample are similar, with images obtained via filtering with a universal cumulative threshold value exhibiting slightly less noise.

Since the universal threshold values effectively mark the end point for acquisition of measured secondary ion signals for sample 150, after suitable threshold values have been determined by controller 114, they can be used by controller 114 in a predictive manner to determine information about sample 150 prior to crossing the thresholds.

In some embodiments, for example, controller 114 can use the determined threshold values and measured secondary ion signals at each location of incidence 124 to determine a thickness of sample 150 remaining at each location. Referring to the plots showing cumulative ion counts on the right side of FIG. 14, it is evident that as sample 150 is exposed to primary ion beam 116, the cumulative measured ion count for many different secondary ions approaches the corresponding cumulative threshold value in a relatively reproducible, monotonic fashion. As such, for a particular total dwell time and ion dose at each location of incidence 124, controller 114 can determine based on the calibration data of FIG. 14 approximately what fraction of sample 150 remains at each location. Furthermore, with calibration information corresponding to the initial thickness of sample 150 at each location, an absolute estimate of the remaining thickness of sample 150 at each location as a function of time can be determined by controller 114.

Controller 114 can also determine other information about the expected rate at which sample 150 will be ablated by primary ion beam 116. For example, by first determining the fraction of sample 150 that has been ablated by primary ion beam 116, the total dwell time, and the total ion dose delivered by the primary ion beam to-date, controller 114 can estimate the prospective ion dose to complete ablation of sample 150 at each location of incidence 124. If the prospective ion dose is to be delivered via multiple exposures at a particular location of incidence 124 of sample 150 to primary ion beam 116, controller 114 can also estimate the number of additional exposures that will lead to complete ablation of sample 150 at the particular location of incidence.

After determining such information, in certain embodiments, controller 114 can modify exposure pattern 400 to take account of the information. For example, at each spatial location within exposure pattern 400, controller 114 can optionally adjust one or more ion beam exposure parameters, including the ion current of primary ion beam 116, the dwell time associated with each spatial location, and the total ion dose delivered to each spatial location, and the number of subsequent visits by primary ion beam 116 to each of the spatial locations of exposure pattern 400. These adjustments are performed prospectively by controller 114 to ensure that secondary ion signals at each spatial location are measured as long as a portion of sample 150 remains at each spatial location, and that measurement of secondary ion signals at each location is discontinued approximately when the last remaining portion of sample 150 at each location is ablated by primary ion beam 116.

### (vii) Hardware and Software Implementations

As discussed above, any of the steps and functions described herein can be executed by controller 114. In general, controller 114 can include a single electronic processor, multiple electronic processors, one or more integrated circuits (e.g., application specific integrated circuits), and any combination of the foregoing elements. Software- and/or hardware-based instructions are executed by controller 114 to perform the steps and functions discussed herein. Each set of software-based instructions, embodied as a software program stored on a tangible, non-transient storage medium (e.g., an optical storage medium such as a CD-ROM or DVD, a magnetic storage medium such as a hard disk, or a persistent solid state storage medium) or device, can be implemented in a high-level procedural or object-oriented programming language, or an assembly or machine language.

Controller 114 can include, or be connected to, a data storage system (including memory and/or storage elements), at least one input device, and at least one output device, such as a display. Controller 114 can display any of the information described herein on the at least one output device, and can receive information and instructions from, and transmit information to, the least one input device, including any of the information and instructions described herein.

### Other Embodiments

A number of embodiments have been described. Nevertheless, it will be understood that various modifications may be made without departing from the spirit and scope of the disclosure. Accordingly, other embodiments are within the scope of the following claims.

What is claimed is:

1. A method of generating ions from a biological sample, the method comprising:
exposing multiple regions of a biological sample on a substrate in succession to an ion beam to generate charged particles from each region, wherein the biological sample is labeled with at least one mass tag;
for each exposed region, analyzing the plurality of charged particles to identify a deviation from a reference distribution of charged particles; and
for each exposed region for which a deviation is identified, adjusting at least one exposure parameter of the ion beam based on the analysis of the plurality of charged particles to modify exposure of the sample to the ion beam.

2. The method of claim 1, wherein analyzing the plurality of charged particles comprises measuring signal peaks corresponding to the charged particles and determining information associated with at least some of the signal peaks, and wherein the information comprises at least one of:
quantitative information comprising at least one of a peak amplitude, a peak area, and a charged particle count associated with each of the at least some of the signal peaks;
at least one of a mass-to-charge ratio and a quantity related to a mass-to-charge ratio associated with each of the at least some of the signal peaks; and
an identity of a charged particle associated with each of the at least some of the signal peaks.

3. The method of claim 2, further comprising, for each exposed region, identifying that a deviation from the reference distribution exists if the quantitative information differs from the reference distribution.

4. The method of claim 3, further comprising, for each exposed region, identifying that a deviation from the reference distribution exists if at least one of:
an element of the quantitative information exceeds a threshold value of the reference distribution;
the charged particles comprise ions generated from the substrate; and
the charged particles comprise ions generated from a coating on the substrate.

5. The method of claim 4, further comprising, for each exposed region, identifying that a deviation from the reference distribution exists if an element of the quantitative information is less than a threshold value of the reference distribution.

6. The method of claim 2, further comprising, for each exposed region, identifying that a deviation from the reference distribution exists if a peak associated with a type of charged particle generated from the region is not present in the reference distribution, and wherein the peak is associated with ions generated from the substrate or with with ions generated from a coating on the substrate.

7. The method of claim 6, further comprising, for each exposed region, identifying that a deviation from the reference distribution exists if a peak associated with a type of charged particle is present in the reference distribution, but not among the peaks measured for the region, wherein the peak is associated with ions generated from the sample.

8. The method of claim 1, wherein adjusting at least one exposure parameter of the ion beam comprises terminating exposure of the region to the ion beam by at least one of:
directing the ion beam away from the region; and
directing the ion beam to be incident on a beam blocking element.

9. The method of claim 1, wherein adjusting at least one exposure parameter of the ion beam comprises reducing a dwell time of the ion beam on the region in a subsequent exposure of the region to the ion beam.

10. The method of claim 1, wherein adjusting at least one exposure parameter of the ion beam comprises reducing an ion current of the ion beam during exposure of the region to the ion beam.

11. The method of claim 1, wherein adjusting at least one exposure parameter of the ion beam comprises reducing an ion current of the ion beam during a subsequent exposure of the region to the ion beam.

12. The method of claim 1, wherein the charged particles comprise secondary electrons generated from one or more of the sample and the substrate, and wherein analyzing the plurality of charged particles comprises:
measuring a signal peak associated with the secondary electrons and determining quantitative information about a secondary electron yield from the signal peak; and
comparing the quantitative information to a threshold value for the secondary electron yield from the reference distribution to determine whether a deviation from the reference distribution exists.

13. The method of claim 1, wherein the charged particles comprise ions generated from the sample, and wherein analyzing the plurality of charged particles comprises:
measuring one or more signal peaks associated with the ions generated from the sample and determining quantitative information about a total ion yield from the sample from the one or more signal peaks; and
comparing the quantitative information to a threshold value for the total ion yield from the reference distribution to determine whether a deviation from the reference distribution exists.

14. The method of claim 1, wherein a set of spatial locations of the multiple regions and exposure parameters of the ion beam at each spatial location define a first exposure sequence for the sample, the method further comprising adjusting the at least one exposure parameter of the ion beam for each exposed region for which a deviation is identified to generate a second exposure sequence for a subsequent exposure of the sample to the ion beam.

15. The method of claim 14, wherein the second exposure sequence comprises fewer spatial locations on the sample than the first exposure sequence.

16. The method of claim 15, wherein all spatial locations of the second exposure sequence are common to the first exposure sequence.

17. The method of claim 14, wherein the second exposure sequence comprises, at one or more spatial locations common to the first exposure sequence, ion beam dwell times that are reduced relative to corresponding dwell times of the first exposure sequence.

18. The method of claim 14, wherein the second exposure sequence comprises, at one or more spatial locations common to the first exposure sequence, ion beam currents that are reduced relative to corresponding ion beam currents of the first exposure sequence.

19. The method of claim 14, further comprising, for each exposed region for which a deviation is identified, determining information about a thickness of the sample in the region based on the deviation from the reference distribution, and generating the second exposure sequence based on the thickness information.

20. The method of claim 19, further comprising determining information about an additional ion beam exposure dose for the region that will lead to elimination of the sample from the region.

21. The method of claim 19, further comprising determining information about an additional number of exposures of the region to the ion beam that will lead to elimination of the sample from the region.

22. The method of claim 1, wherein the biological sample is labeled with multiple, different types of mass tags, and wherein each of the types of mass tags comprises an antibody-conjugated lanthanide element.

23. The method of claim 1, wherein the biological sample comprises at least one of a tissue sample and an array of single cells.
